(19) United States
(12) Patent Application Publication
(10) Pub. No.: US 2020/0277675 A1
(43) Pub. Date: Sep. 3, 2020

(54) BIOMARKERS FOR PREDICTING RESPONSE TO CLOSTRIDIUM DIFFICILE

(71) Applicant: BIOSYS UK LIMITED, London Greater London (GB)

(72) Inventors: Michael Pfaffl, Munich (DE); Maria Hillreiner, Munich (DE); Heike Kliem, Munich (DE)

(21) Appl. No.: 16/643,757

(22) PCT Filed: Sep. 5, 2018
(86) PCT No.: PCT/EP2018/073837

§ 371 Date: Mar. 2, 2020

(30) Foreign Application Priority Data
Sep. 5, 2017 (GB) 1714220.9

(51) Int. Cl. C12Q 1/6876; A61K 39/08

(52) U.S. Cl. CPC C12Q 1/6876; A61K 39/08; C12Q 2600/124; C12Q 2600/158; C12Q 2600/106

(57) ABSTRACT

The invention relates to methods for identifying/selecting cows for immunisation and to the use of markers for such methods. The methods involve interrogation of cells from a cow, and the use of markers in those cells to identify cows for immunisation. Cows are selected for immunisation by observing the change in expression of markers resulting from stimulating a cell obtained from a cow with C. difficile and/or a C. difficile specific antigen.

### TECHNICAL FIELD

The invention relates to methods for identifying/selecting cows for immunisation and to the use of markers for such methods.

### BACKGROUND

Clostridium difficile (C. diff) is a widespread hospital germ that causes severe antibiotic associated gastroenteritis in humans especially in industrialized countries [1,2]. C. diff is a gram-positive enterotoxic, spore building pathogen that due to its acidic resistance is able to overcome the acidic environment of the stomach [1,3]. Clostridium difficile infection (CDI) is a global problem especially affecting patients in hospitals and long-term health care facilities. The primary reservoir of the pathogen are asymptomatic carriers and contaminated surfaces that are a severe issue especially in hospitals and nursing homes [4]. In particular C. difficile invention causes severe antibiotic associated gastroenteritis in humans especially in industrialized countries.

The progression of the disease is quite diverse, ranging from mild diarrhea to severe life threatening pseudomembranous colitis (PMC) [1,3,5]. Symptoms of CDI include diarrhoea, fever, and inflammation of the bowel sometimes including pseudomembranous colitis (PMC) or fulminant colitis (“mega colon”).

Antibodies for the treatment of Clostridium difficile infection (CDI) have been demonstrated to be effective in the research and clinical environments, and several studies have shown the utility of lacteal antibodies in the treatment of C. difficile infections (see, for, example Van Dissel et al. (2005) [9].

It is therefore desirable to develop an immune milk, enriched with naturally derived polyclonal immunoglobulin A (IgA) against C. difficile.

### SUMMARY OF THE INVENTION

The present invention relates generally to methods of selecting a cow for immunisation and to methods of identifying cow for immunisation.

The invention is based on work investigating whether animals can be pre-selected concerning their ability to produce high amounts of specific immunoglobulins upon vaccination with a C. difficile vaccine. The investigation used gene expression profiling to identify molecular markers within the innate immune system of cows.

As the production and the application of the immune milk are quite promising, it would be favourable to optimize the specific IgA against C. difficile yield in the milk secretion. Optimization of the immunoglobulin yield in bovine milk used as therapeutic immune milk or whey for the prevention of Clostridium difficile associated diarrhoea (CDAD) in humans is of great importance in case of economic efficiency of the product. It is already known that dairy cows show diverse immune responses upon vaccination, resulting in a variable immunoglobulin yield in blood or milk. Therefore, it is desirable to pre-select the cows concerning their ability to produce and secrete high yields of specific immunoglobulins. Elucidation of the underlying gene expression network may facilitate the selection of the animals before they are used for immune milk production.

The inventors have discovered markers which are predictive of an ability to produce high amounts of specific immunoglobulins upon vaccination. This allows optimisation of antibody yield by selection of cows which are predisposed to produce and secrete high levels of specific immunoglobulin.

In particular, the inventors have identified genes which are differentially up or down-regulated in high responder cows as compared to low responder cows.

Accordingly, the methods of the invention involve interrogation of cells from a cow, and the use of markers in those cells to identify cows for immunisation. Using the methods herein, cows are selected for immunisation by observing the change in expression of markers resulting from stimulating a cell obtained from a cow with C. difficile and/or a C. difficile specific antigen.

In the present invention genes of interest serve as markers or biomarkers. mRNA levels in the cell can be used to determine expression.

Generally methods of the present invention, comprise the steps:
(a) determining expression level of a plurality of genes of interest in a cell obtained from a cow
(b) stimulating the cell with C. difficile and/or a C. difficile specific antigen
(c) determining the expression of the plurality of genes of interest.

The methods may comprise a step of comparing the expression level of a plurality of genes of interest in step (a) with the expression level in step (c). A change in expression between step (a) and (c) of a gene of interest may be indicative of a high responder cow.

In particular, a significant change in expression of a gene of interest between steps (a) and (c) may be indicative of a high responder cow.

In particular, the change in expression may be at least about that shown in supplementary table 2 for any given gene. The change in gene expression may be at least about the mid-point between the change in expression of a high responder and a low responder shown in supplementary table 2. In particular, at time-points where the increase or decrease in expression of a gene shown in supplementary table 2 is greater for the high responder cow, a change in gene expression that is at least the mid-point between change in expression of a high responder and a low responder shown in supplementary table 2 is indicative of a high responder cow. Additional subsets of genes are given elsewhere herein.

For obtaining these values from supplementary table 2, ‘6 h’ is representative of an early time point, ‘24 h’ is representative of an intermediate time point, and ‘72 h’ is representative of a late time point.

The fold-change in expression as deducible from supplementary table 2, may be applied as the fold-change to any of the methods disclosed herein.

For example, where the change in gene expression is taken to be at least about that in table 2 for TLR2, the change in expression of TLR2 at an early time point of at least about 1.51, would be indicative of a high responder cow. Similarly, where the change in gene expression is taken to be at least about the midpoint between the change in expression of a high responder and a low responder, a change in TLR2 expression at an early time-point of at least about 1.325 would be indicative of a high responder cow.

In particular, the change in expression may be at least 1.5-fold or at least two-fold, for example a two-fold increase in expression or a two-fold decrease in expression. Accordingly, the method may comprise a further step:
(d) selecting a cow if there is an at least two fold increase or decrease in expression of the plurality of genes of interest.

As used herein a two-fold decrease in gene expression refers to reduction in expression by half. In other words the expression level is dived by 2. In other words, an indication of a fold-change of 0.5 represents a two-fold decrease in expression. Similarly a 1.5-fold decrease in expression would be a reduction equivalent to dividing the expression level by 1.5.

In the context of the invention ‘high responder cows’ are cows which, having been immunized, can produce a threshold amount of C. difficile specific IgA in their secreted milk. For example, a high responder cow may produce at least 8 μg/ml of C. difficile specific IgA in their secreted milk post immunisation. For example, a high responder cow may produce significantly more C. difficile specific IgA than a low responder cow treated with the same immunisation protocol. The milk may also contain other Ig subtypes.

Similarly, ‘low responder cows’ are cows which, having been immunized, produce below a threshold amount of C. difficile specific IgA in their secreted milk. A low responder cow may produce less than 8 μg/ml of C. difficile specific IgA in their secreted milk post immunisation. For example, a low responder cow may produce less than about 7 μg/ml of C. difficile specific IgA in their secreted milk post immunisation. For example, a low responder cow may produce significantly less C. difficile specific IgA than a high responder cow treated with the same immunisation protocol.

For this purpose, the amount of C. difficile specific IgA in the milk in μg/ml may be taken as an average (mean) of the amount measured at multiple time points. For example, the amount of C. difficile specific IgA may be measured across the vaccination/immunisation schedule. The amount of IgA may be measured every one or two weeks throughout the vaccination protocol, for example, and the mean value taken. For example, the amount of IgA may be measured every one to two weeks from week 3 onwards, for example, in weeks 3-18, or 4-30 of the immunisation protocol.

The levels of antibodies can be determined by ELISA, for example.

Comparison of the change in gene expression between steps (a) and (c) with the change in expression of the same genes of interest in a cell from a cow already determined to be a high responder or a low responder cow may be carried out. Accordingly, the methods may comprise the step of:
(d) comparing the change in expression of the genes of interest between step (a) and (c) with the expression change for the same genes of interest in a cow already determined to be a high responder and a cow already determined to be low responder cow, and
(e) selecting a cow if the change in expression levels of the genes determined in step (d) is more similar to the change for a high responder cow than a low responder cow.

The change in gene expression may be compared to the change in expression determined using steps (a)-(c) for a high responder cow and a low responder cow. In particular, the change in expression may be compared to that seen for a group of cows already determined to be high responder cows and a group of cows already determined to be low responder cows.

The changes in gene expression may be compared using mathematical methods suitable for determining correlations and dependencies. For example, Pearson's correlation, Spearman's rank or Kendall tau rank may be used.

Selection of cows for immunisation according to the methods described may also be referred to as selection of cows predisposed to be high responder cows, or selection of cows which display markers indicative of high responder cows.

Accordingly, the present invention provides methods of selecting or identifying a cow for immunisation, the method comprising
(a) determining expression level of a plurality of genes of interest in a cell obtained from a cow
(b) stimulating the cell with C. difficile and/or a C. difficile specific antigen
(c) determining the expression of the plurality of genes of interest
(d) selecting a cow if there is an at least two-fold increase or decrease in expression of the plurality of genes of interest.

The present invention also provides methods of selecting or identifying a cow for immunisation, the method comprising
(a) determining expression level of a plurality of genes of interest in a cell obtained from a cow
(b) stimulating the cell with C. difficile and/or a C. difficile specific antigen
(c) determining the expression of the plurality of genes of interest
(d) comparing the change in expression of the genes of interest between step (a) and (c) with the expression change for the same genes of interest in a cow already determined to be a high responder and a cow already determined to be low responder cow, and
(e) selecting a cow if the change in expression levels of the genes determined in step (d) is more similar to the change for a high responder cow than a low responder cow.

In this method, a cow may be selected if an at least two-fold increase or decrease in expression of the plurality of genes of interest is also found.

The methods may be for selecting or identifying a cow for immunisation with a C. difficile vaccine.

The invention also provides a method of determining whether a cow is predisposed to produce high levels of C. difficile specific IgA in milk.

The invention also provides a method for predicting whether a cow is a high responder cow, comprising the steps:
(a) determining expression level of a plurality of genes of interest in a cell obtained from a cow,
(b) stimulating the cell with C. difficile and/or a C. difficile specific antigen
(c) determining the expression of the plurality of genes of interest,
where an at least two-fold increase or decrease in expression between step (a) and (c) of a gene of interest is predictive of a cow being a high responder cow.

The invention also provides a method of identifying cows for immunisation in a population of cows, or selecting cows from a population of cows for immunisation.

The present invention also provides a method of identifying cows predisposed to be high responder cows in a population of cows, or selecting cows predisposed to be high responder cows from a population of cows.

The invention also provides the use of a panel of biomarkers for identifying cells for immunisation. The markers may be used according to the methods described herein.

Also provided are isolated antibodies, produced by a cow selected for vaccination according to the methods of the invention, and vaccinated with a C. diff specific antigen. The antibodies may be a purified polyclonal mixture. The antibodies may be specific to C. difficile toxin A, and/or C. difficile toxin B, and/or another C. difficile cellular antigen.

### Markers

In the context of the present invention the ‘markers’ or ‘biomarkers’ are genes of interest which can be used to distinguish high responder cows from low responder cows.

Expression of these genes, or more specifically change in expression of these genes, is indicative of whether a cow would be a ‘high responder’ to Clostridium difficile vaccination. In other words, the markers are predictive of high levels of secretion of anti-C. difficile specific antibodies in cow milk.

Generally, a plurality of markers or genes of interest are used to select or identify a cow for immunisation. Using a plurality of genes a signature change in gene expression can be seen, which is indicative of a high responder cow.

Generally the markers identified indicate that high responder animals have a stronger and quicker induction of the innate immune response. Accordingly, the genes of interest may be linked to the innate immune system. In particular, genes of interest include genes encoding components of the TLR pathway, Chemokines, Inflammatory cytokines, Acute phase proteins and danger associated molecular pattern molecules, Antimicrobial peptides, Apoptosis, Scavenger receptor, JAK-STAT signalling, MAPK signalling.

In particular, a gene of interest may be as set forth in Supplementary Table 2.

Further details of the genes of interest are provided in Supplementary Table 1, including the full gene names, NCBI reference numbers, each of which identifies an NCBI reference sequence record which provides, inter alia, the Bos taurus mRNA sequence for the respective gene. The entire contents of each NCBI reference sequence as retrievable on 24 Jul. 2017 using the respective NCBI reference sequence number from Supplementary Table 1 is incorporated herein by reference.

In particular, a gene of interest may encode a component of the TLR-pathway (e.g. LY96, CD14, TIRAP and RELA), the chemokines (e.g. CXCL8, CCL5, CXCL5), inflammatory cytokines (e.g. IL6, IL1-A), antimicrobial peptides (e.g. LYZ1, LPO) and danger associated molecular pattern molecules (e.g. S100A9, S100A12)

Genes of interest include 1 or more of the genes:

LF, TGFB1, CASP3, TIRAP, CXCL5, TLR2, SAA3, NOS2, CXCL8, AKT1, IRF3, C3, TNFR2, FOS, CXCL3, CCL5, CCR7, LY96, TRAF6, MYD88, STAT2, TLR4, TNFRSF1A, IL13RA, CASP8, MAPK8, IRAK1, S100A12, BCL-X1, CCL20, NOD2, LPO, RELA, TNFα, LYZ1, BAX, IL6, CD40, WNT4, IL1-A, CD68, MMP1, IRAK4, FAS, CD14, S100A9, BCL2, CYP1B1, LBP, MX1, and MX2.

The genes of interest may optionally further include immunoglobulin related genes FcRN and/or pIGR.

In the methods of the invention, expression of 2, 3, 4, 5, 6, 7, 8, 9, 10, 11, 12, 13, 14, 15, 16, 17, 18, 19, 20, 25, 30, 35, 40, 45 or more of these genes may be determined in the determining steps. For example the expression of 3 or more, 5 or more, 8 or more, 10 or more, or 12 or more of the genes of interest may be determined. For example, the expression of 3-5, 3-10, 3-15, 5-12, 5-15, 5-20, 5-30, 5-40, 10-40 or all of the genes of interest may be determined. For example, expression of at least 3, at least 5, at least 8, at least 10, or at least 12 of the genes of interest may be determined.

In some embodiments, expression of the 3 genes LY96, CD14 and TIRAP may be determined. As described further herein (see Table 1), the statistical significance between the low responder and high responder groups, as assessed by t-test p value, was found to be greatest (i.e. lowest p value) for LY96, CD14 and TIRAP. Each exhibited a p≤0.001 at 24 or 72 hours low vs high. This panel of 3 genes therefore represents a strong gene expression discriminator of low and high responders.

The gene expression level after stimulation may be determined after up to 96 hours of stimulation. In other words the gene expression level in the cell may be determined after incubation of the cell with C. difficile or a C. difficile specific antigen for up to 96 hours.

The various genes of interest may be differentially expressed at different time-points following stimulation of the cell. Accordingly, the expression of the plurality of genes of interest may be conducted at one or more time points after stimulation of the cell. In other words, the cell may be stimulated with C. difficile or a C. difficile antigen for different periods of time before determining expression of a gene of interest. This enables detection of gene expression changes over different time periods.

‘Early’ response and early gene expression changes (increases or decreases) may be detected after stimulation with a C. difficile antigen for up to 22 hours, for example, about 5 minutes-22 hours.

Determining gene expression at an ‘early’ time point is therefore up to 22 hours after the C. difficile has been added. For example, early gene expression may be determined after about 1-16 hours, about 1-12 hours, about 3-9 hours, about 4-8 hours, for example after about 6 hours of stimulation with a C. difficile antigen.

‘Intermediate’ response and intermediate gene expression changes (increases or decreases) may be detected after stimulation with a C. difficile antigen for about 22-48 hours. Determining gene expression at an ‘intermediate’ time point is therefore 22-48 hours after the C. difficile has been added. For example, intermediate gene expression may be determined after about 24-42 hours, about 22-30 hours, about 22-28 hours, for example after about 24 hours of stimulation with a C. difficile antigen.

‘Late’ response and late gene expression changes may be detected after stimulation with a C. difficile antigen for about 48-96 hours. Determining gene expression at a ‘late’ time point is therefore 48-96 hours after the C. difficile has been added. For example, late gene expression may be determined after about 52-84 hours, about 66-78 hours, about 68-76 hours, for example after about 72 hours of stimulation with a C. difficile antigen.

Expression of a gene of interest may be determined at multiple time points after stimulation. For example expression may be determined at 1, 2 or all of an early, intermediate and a late time point. In other words, the expression of a gene of interest may be tracked over time, for example between early, intermediate and late time points.

In some embodiments, a cow is identified or selected if a gene of interest shows increased or decreased expression after stimulation for at two or three of an early, intermediate and late time point, for example, a gene of interest may have increased or decreased expression during all three time-points.

In particular, a cow may be selected if the cell shows a change in gene expression, at an early time point, of one or more of the following genes:

SAA3, LF, C3, TIRAP, CXCL5, CXCL3, CXCL8, TRAF6, RELA, CD14, CCL5, IL6, FAS, CASP3, BCL-2, CD68, CD40, MAPK8, TGFB1, STAT2, TLR4, AKT1, TNFRSF1A, WNT4, IRF3, TNFR2, FOS, TLR2, NOS2, LBP, MX1.

Accordingly, in some embodiments the expression of one or more genes of interest selected from: SAA3, LF, C3, TIRAP, CXCL5, CXCL3, CXCL8, TRAF6, RELA, CD14, CCL5, IL6, FAS, CASP3, BCL-2, CD68, CD40, MAPK8, TGFB1, STAT2, TLR4, AKT1, TNFRSF1A, WNT4, IRF3, TNFR2, TLR2, NOS2, LBP and FOS is determined;
wherein the cow is selected if there is an at least two-fold increase or decrease in expression of the one or more genes of interest after stimulation with C. difficile and/or a C. difficile specific antigen for up to 22 hours.

Accordingly, in some embodiments the expression of one or more genes of interest selected from: SAA3, LF, C3, TIRAP, CXCL5, CXCL3, CXCL8, TRAF6, RELA, CD14, CCL5, IL6, FAS, CASP3, BCL-2, CD68, CD40, MAPK8, TGFB1, STAT2, TLR2, TLR4 AKT1, TNFRSF1A, WNT4, IRF3, TNFR2, NOS2, LBP and FOS is determined, and the change in expression of these genes after stimulation with C. difficile and/or a C. difficile specific antigen for up to 22 hours is compared with the expression change for the same genes in a cow already determined to be a high responder and a cow already determined to be low responder cow, wherein a cow is selected if the change in expression levels of the genes determined in step is more similar to the change for a high responder cow than a low responder cow.

The change in gene expression for the high and low responder cows is measured at the same time point (after the same period of stimulation) as for the cow being tested.

In particular, expression of one or more, or 3 or more, or all of the following genes may be determined: SAA3, LF, C3, TIRAP, CXCL5, CXCL3, CXCL8, TRAF6, RELA.

In particular, a cow may be selected if the cell shows an increase in gene expression, at an early time point, of one or more of the following genes: LF, CASP3, TIRAP, CXCL5S, TLR2, SAA3, NOS2, CXCL8, AKT1, IRF3, C3, TNFR2, LBP, MX1, LBP, CXCL3 and FOS.

In particular, a cow may be selected if the cell shows a decrease in gene expression, at an early time point, of one or more of the following genes: CD14, IL6, FAS, BCL-2, CD68, WNT4.

In particular, expression of one or more, or 3 or more, or all of the following genes may be determined: CXCL8, CXCL5, TIRAP, RELA, FAS, IL6 CASP3, CD68, CD40, MAPK8. For example, expression of one or more, or 3 or more, or all of the following genes may be determined: TIRAP, FAS, IL6 CASP3, CD68, CD40, MAPK8. For example, expression of one or more, or 3 or all of the following genes may be determined: CXCL8, CXCL5, TIRAP, RELA. In particular, expression of one or more, or 3 or more, or all of the following genes may be determined: TRAF6, LF, TGFBI, RELA, CASP3, TIRAP, CXCL5, STAT2, SAA3, CXCL8, TLR4, AKT1, TNFRSF1A, WNT4, IRF3, C3, TNFR2, FOS, CXCL3.

In particular, a cow may be selected if the cell shows increased or decreased gene expression, at an intermediate time point, of one or more of the following genes: CCL5, CCR7, LY96, TRAF6, MYD88, STAT2, TLR4, TLR2, CD14, TIRAP, THFRSF1A, IL13RA, CASP8, CASP3, MAPK8, IRAK1, IRAK4, RELA, IL6, FAS, BAX, CD68, CD40, MMP1, AKT1, NOS2, LBP, MX1, MX2, TGFB1.

Accordingly, in some embodiments the expression of one or more genes of interest selected from: CCL5, CCR7, LY96, TRAF6, MYD88, STAT2, TLR4, TLR2, CD14, TIRAP, THFRSF1A, IL13RA, CASP8, CASP3, MAPK8, IRAK1, IRAK4, RELA, IL6, FAS, BAX, CD68, CD40, MMP1, AKT1, TGFB1 NOS2, LBP, MX1 and MX2 is determined;
wherein the cow is selected if there is an at least two-fold increase or decrease in expression of the one or more genes of interest after stimulation with C. difficile and/or a C. difficile specific antigen for 22-48 hours.

Accordingly, in some embodiments the expression of one or more genes of interest selected from: CCL5, CCR7, LY96, TRAF6, MYD88, STAT2, TLR4, TLR2, CD14, TIRAP, THFRSF1A, IL13RA, CASP8, CASP3, MAPK8, IRAK1, IRAK4, RELA, IL6, FAS, BAX, CD68, CD40, MMP1, AKT1, TGFB1 NOS2, LBP, MX1 and MX2 is determined, and the change in expression of these genes after stimulation with C. difficile and/or a C. difficile specific antigen for 22-48 hours is compared with the expression change for the same genes in a cow already determined to be a high responder and a cow already determined to be low responder cow, wherein a cow is selected if the change in expression levels of the genes determined in step is more similar to the change for a high responder cow than a low responder cow.

The change in gene expression for the high and low responder cows is measured at the same time point (after the same period of stimulation) as for the cow being tested.

In particular, expression of one or more, or 3 or more, or all of the following genes may be determined: LY96, CD14, TIRAP, IRAK4, RELA, CCL5, IL13RA, FAS, CASP8, CASP3, BAX, CD68, CD40, STAT2, MAPK8, MMP1, AKT1. In particular, expression of one or more, or 3 or more, or all of the following genes may be determined:

LY96, CD14, TIRAP, RELA, CCL5, IL6. In particular, expression of one or more, or 3 or more, or all of the following genes may be determined: LY96, MYD88, IRAK1, CCL5, MAPK8.

In particular, expression of one or more, or 3 or more, or all of the following genes may be determined: CCL5, CCR7, LY96, MYD88, TLR2, NOS2, LBP, MX1, MX2, MAPK8, IRAK1.

In particular, a cow may be selected if the cell shows a decrease in gene expression, at an intermediate time point, of one or more of the following genes: NOS2, LBP, MX1, MX2, BAX.

In some embodiments the expression level of 1, 2, 3, or all of the following genes may be determined, and a cow selected if there is an at least two-fold decrease in expression after stimulation with C. difficile and/or a C. difficile specific antigen for 22-48 hours: NOS2, LBP, MX1, MX2.

In particular, a cow may be selected if the cell shows increased or decreased gene expression, at a late time point, of one or more of the following genes: IL6, IL1-A, IL13RA, S100A12, S100A9, LYZ1, LPO, CD14, FAS, CASP8, BAX, CD68, CD40, MMP1, NOD2, BCL-xL, CCL20, TNFα, IRAK4, CYP1B1, LY96, TIRAP, CXCL8, IL13RA, BCL2, WNT4, RELA, MX2.

Accordingly, in some embodiments the expression of one or more genes of interest selected from: IL6, IL1-A, IL13RA, S100A12, S100A9, LYZ1, LPO, CD14, FAS, CASP8, BAX, CD68, CD40, MMP1, NOD2, BCL-xL, CCL20, TNFα, IRAK4, CYP1B1, LY96, TIRAP, CXCL8, IL13RA, BCL2, RELA, MX2 and WNT4 is determined;
wherein the cow is selected if there is an at least two-fold increase or decrease in expression of the one or more genes of interest after stimulation with C. difficile and/or a C. difficile specific antigen for 48-96 hours.

Accordingly, in some embodiments the expression of one or more genes of interest selected from: IL6, IL1-A, IL13RA, S100A12, S100A9, LYZ1, LPO, CD14, FAS, CASP8, BAX, CD68, CD40, MMP1, NOD2, BCL-xL, CCL20, TNFα, IRAK4, CYP1B1, LY96, TIRAP, CXCL8, IL13RA, BCL2, RELA, MX2 and WNT4 is determined, and the change in expression of these genes after stimulation with C. difficile and/or a C. difficile specific antigen for 48-96 hours is compared with the expression change for the same genes in a cow already determined to be a high responder and a cow already determined to be low responder cow, wherein a cow is selected if the change in expression levels of the genes determined in step is more similar to the change for a high responder cow than a low responder cow.

The change in gene expression for the high and low responder cows is measured at the same time point (after the same period of stimulation) as for the cow being tested.

In particular, expression of one or more, or 3 or more, or all of the following genes may be determined: S100A9, S100A12, IL6, IL1-A, LYZ1, LPO, TIRAP, LY96, CXCL8, CD14. In particular, expression of one or more, or 3 or more, or all of the following genes may be determined: LY96, CD14, IL6, S100A9, S100A12, LYZ1, LPO, BCL-2, CD40, MMP1, NOD2. In particular, expression of one or more, or 3, or all of the following genes may be determined: S100A9, S100A12, LYZ1, LPO. In particular, expression of one or more, or 3, or all of the following genes may be determined: S100A12, BCL-xL, CCL20, NOD2, LPO, RELA, TNFα, LYZ1, BAX, IL6, CD40, WNT4, IL1-A, CD68, MMP1, IRAK4, MX2, FAS, CD14, S100A9, BCL2, CYP1B1, CD68.

In some embodiments a cow is selected if:
(a) there is an at least two-fold change in gene expression of one or more of SAA3, LF, C3, TIRAP, CXCL5, CXCL3, CXCL8, TRAF6, RELA, CD14, CCL5, IL6, FAS, CASP3, BCL-2, CD68, CD40, MAPK8, TGFB1, STAT2, TLR4, AKT1, TNFRSF1A, WNT4, IRF3, C3, TNFR2, FOS, NOS2, LBP and MX1 after stimulation with C. difficile and/or a C. difficile specific antigen for up to 22 hours; and/or
(b) there is an at least two-fold change in gene expression of one or more of CCL5, CCR7, LY96, TRAF6, MYD88, STAT2, TLR4, TLR2, CD14, TIRAP, THFRSF1A, IL13RA, CASP8, CASP3, MAPK8, IRAK1, IRAK4, RELA, IL6, FAS, BAX, CD68, CD40, MMP1, AKT1, NOS2, LBP, MX1, MX2, TGFB1 after stimulation with C. difficile and/or a C. difficile specific antigen for 22-48 hours; and/or
(c) there is an at least two-fold change in gene expression of one or more of IL6, IL1-A, IL13RA, S100A12, S100A9, LYZ1, LPO, CD14, FAS, CASP8, BAX, CD68, CD40, MMP1, NOD2, BCL-xL, CCL20, TNFα, IRAK4, CYP1B1, LY96, TIRAP, CXCL8, IL13RA, BCL2 RELA, MX2 and WNT4 after stimulation with C. difficile and/or a C. difficile specific antigen for 48-96 hours.

For example, a cow may be selected if there is an at least two-fold increase or decrease in one or more genes in (a) and (b), or and at least two-fold increase or decrease in one or more genes in (b) and (c), or at least two-fold increase in one or more genes in (a), (b) and (c).

In particular, an at least two-fold increase or decrease in gene expression after stimulation is indicative of being a high responder cow. Accordingly, a cow may be selected if there is an at least two-fold increase or decrease in expression of a gene of interest. A cow may be selected if there is an at least three- or four-fold increase in expression of a gene of interest.

Where the expression level of more than one gene of interest is determined, a cow may be selected if at least half of the genes of interest show an at least two-fold increase or decrease in expression. A cow may be selected if at least 3 in 5, 2 in 3, 3 in 4 or all of the genes of interest for which expression is determined show an at least two-fold increase or decrease in expression. A cow may be selected if at least 50%, 60%, 70%, 75%, 80%, 90% or 95% of the genes of interest for which expression is determined have an at least two-fold increase or decrease in expression following stimulation.

Again, the threshold for selection may be set higher, for example an at least 2.5-fold, 3-for 4-fold or 5-fold increase or decrease in expression.

### Methods of Identifying Markers

Expression of the one or more genes of interest can be evaluated using methods well-known in the art. For example, the relative level of mRNA can be determined using “quantitative” amplification methods. One well known example, quantitative PCR (qPCR), involves simultaneously co-amplifying a control sequence whose quantities are known to be unchanged between control and subject samples. This provides an internal standard that may be used to calibrate the PCR reaction.

The level of gene expression may be determined by reverse transcription-qPCR (RT-qPCR). Alternatively level of gene expression may be determined by a mRNA microarray, any hybridisation based method, and PCR based method or any sequencing based method.

Methods of quantifying gene expression are well known to the skilled person, including relative quantification techniques [23, 35-37]. More details about a of gene expression approach can be found at http://relative.gene-quantification.info/.

### Cows

The cows for use in the methods of the present inventions are female cattle of the species Bos taurus. Preferably the cows are dairy cows. Breeds of dairy cattle are known to the skilled person and include: Holstein, Friesian, Holstein-Friesian, Brown Swiss, Guernsey, Ayrshire, Jersey, Red and White, Milking Shorthorn, Bus̆a, Canadienne, Dairy Shorthorn, Dexeter, Illawarra, Irish Moiled, American Milking Devon, Norwegian Red, Dutch Belted, Estonian Red, Fleckvieh, Girolando, Kerry, Lineback, Muese Rhine Issel, Montbeliarde, Normande, Randall, Sahiwal and Red Poll or crossbreeds of any of these breeds. In particular the cows may be Brown Swiss cows.

Although the methods are described throughout in the context of cows, the methods of the invention could also be applied to other milk-producing mammals, in particular other mammals from the Bovidae family such as goats.

### Cells

The cells used in the methods of the present invention may be any cells which show distinct differences in gene expression of effector molecules of the innate immune system. In particular, immune cells may be used.

Primary bovine mammary epithelial cells (pbMEC) are quite important in the case of promotion of innate immunity and hence activation of adaptive immunity and later on transcytosis and secretion of immunoglobulins into the milk. Other immune cells such as lymphocytes would be expected to respond similarly to pbMEC.

Accordingly, the cells used in the methods may be pbMEC or lymphocytes. pbMEC may be isolated from fresh milk of cows. Lymphocytes may be isolated from blood samples. Lymphocytes or lymphocyte subpopulations may be isolated from blood or milk samples.

The methods may also comprise a step of obtaining a cell from a cow. For example, the methods may comprise the steps:
(a) obtaining a cell from the cow,
(b) determining expression level of a plurality of genes of interest in the cell,
(c) stimulating the cell with C. difficile and/or a C. difficile specific antigen,
(d) determining the expression of the plurality of genes of interest, and
(e) selecting a cow if there is an at least two-fold increase or decrease in expression of the plurality of genes of interest.

The present invention also provides methods of selecting or identifying a cow for immunisation, the method comprising
(a) obtaining a cell from the cow,
(b) determining expression level of a plurality of genes of interest in the cell,
(c) stimulating the cell with C. difficile and/or a C. difficile specific antigen,
(d) determining the expression of the plurality of genes of interest, and
(e) comparing the change in expression of the genes of interest between step (a) and (c) with the expression change for the same genes of interest in a cow already determined to be a high responder and a cow already determined to be low responder cow, and
(f) selecting a cow if the change in expression levels of the genes determined in step (d) is more similar to the change for a high responder cow than a low responder cow.

The methods may involve determining expression level of or more genes of interest in a plurality of cells obtained from the cow. For example, a plurality of pbMECs or a plurality of lymphocytes.

### Stimulating Cells

In the methods of the invention, the cells are stimulated with C. difficile and/or a C. difficile specific antigen. Any substance which is able to induce a C. difficile specific immune response is suitable.

The antigen may be Toxin A and/or Toxin B. The C. difficile may be inactivated. Accordingly, in the methods of the invention, the cell may be contacted by one or more of inactivated C. difficile, C. difficile Toxin A and C. difficile Toxin B. For example, the cell may be stimulated by contacted with inactivated C. difficile, or C. difficile Toxins A and B.

The inactivated C. difficile may be formalin inactivated C. difficile.

The C. difficile toxins may be whole, or fragments of the wild-type toxins which are able to induce an immune response.

Selection of a Cow from a Population

The invention also provides a method of identifying cows for immunisation in a population of cows, or selecting cows from a population of cows for immunisation.

The present invention provides a method of identifying cows predisposed to be high responder cows in a population of cows, and methods of selecting cows predisposed to be high responder cows from a population of cows.

The methods of identifying/selecting cows from a population of cows may comprise the steps of:
(a) determining expression level of a plurality of genes of interest in a cell obtained from each cow in the population
(b) stimulating the cells with C. difficile and/or a C. difficile specific antigen
(c) determining the expression of the plurality of genes of interest in the stimulated cells.
(d) selecting a cow if the fold-increase or decrease in expression of each of the plurality of genes of interest has a p value of less than 0.1 as determined by a normal t-test as compared to the mean fold-increase or decrease.

The method may comprise the step of comparing the expression levels of the genes of interest in steps (a) and (c).

A cow is predisposed to be a high responder, or is selected for immunisation if it has a greater absolute change (increase or decrease in expression of a gene of interest than the mean change in expression of the population. A cow may be selected or identified if the fold-change in expression of a gene of interest is greater than 1 standard deviation above the mean fold-change. A cow may be selected or identified if the fold-change in expression of a gene of interest is greater than 2 standard deviations above the mean fold-change.

In particular a cow may be selected or identified if the fold-change in expression of a gene of interest is at least a distinct change as compared to the mean fold-change. Fold-changes having a p value of 0.05≤p≤0.10 as determined by a statistical test are considered distinct. A cow may be selected or identified if the fold-changes in expression of a gene of interest is significant as compared to the mean fold-change. Fold-changes having a p value of p≤0.05 as determined by a statistical test are considered significant. The statistical test may be a t-test, for example a normal t-test. Other suitable tests may be used.

The population of cows may have at least 5 at least 10, at least 15, at least 20, at least 25, at least 30, at least 40, at least 50 or at least 70 cows.

In these methods, the genes of interest are the same as those identified under the heading ‘markers’ above. Similarly, all of the description of the cows, cells, and other aspects of the methods applies equally to the methods of selecting a cow from a population.

In particular, the markers may be detected at the same time points as identified under the heading ‘markers’ above.

### Additional Screening Steps

The methods of identifying and selecting cows described herein may comprise further screening steps. For example, the cows may additionally be screened for disease before being selected for immunisation. Accordingly, the methods may further comprise the step of screening the cows for infection before selecting the cows for immunisation. This step may be carried out before obtaining a cell for stimulation, after the stimulation and gene detection steps, or concurrently with the other steps.

An additional screening step may be carried out on a stool sample from the cows. A cow which has an infection may be de-selected, or not selected for immunisation. For example, a cow which is C. difficile positive, for example in a stool sample, may be excluded from immunisation.

A cow which is positive for other pathogens may also be excluded from immunisation. For example, Staphylococcus, such as Staphylococcus aureus, infection or Streptococcus uberis infection.

### Selected Cows

Cows identified or selected using the methods described herein may be used for production of milk containing C. difficile specific antibodies. As explained above, the use of such polyclonal antibodies is useful for the treatment of C. difficile infection in humans.

Accordingly, the methods described herein may be methods of selecting and immunising a cow and may further comprise the step of:
(i) immunising the selected or identified cow(s) with C. difficile vaccine.

Also provided are methods of obtaining milk containing C. difficile specific antibodies. The methods may therefore comprise the additional step:
(ii) collecting milk from the immunised cows.

Also provided are methods of treatment of an individual having a C. difficile infection, and may also comprise the step of:
(iii) administration of the collected milk to a patient in need thereof.

More specifically, C. difficile-specific antibodies concentrated from the collected milk may be administered to the patient. For example, including C. difficile-specific IgA. C. difficile specific antibodies include antibodies to C. difficile toxins.

Also provided is a C. difficile vaccine for use in a method of immunising a cow, wherein the cow has been identified as being predisposed to be a high responder using the methods described herein.

Also provided is a method of immunising a cow, comprising administering a C. difficile vaccine to a cow that has been identified as being predisposed to be a high responder using the methods described herein.

As described elsewhere herein, cows predisposed to be high responder cows are cows from which cells stimulated with C. difficile and/or a C. difficile specific antigen show the characteristic change in gene expression.

Suitable vaccines include the Clostridium difficile vaccine by IDT Biologika GmbH (Dessau-Rosslau, Germany), though other vaccines are available, for example TGC Biomics vaccine. The vaccine may contain C. diff toxin A, and/or C. diff toxin B, and/or inactivated or ‘killed’ C. diff cells.

### Kits

The invention also provides kits for use in the methods described herein. In other words, the invention provides a kit for selecting a cow for immunisation and for determining whether a cow is predisposed to produce high levels of C. difficile specific IgA.

The kit may comprise specific binding agents for detecting the biomarkers. These specific binding agents may also be referred to as probes. Additionally or alternatively, the kit may comprise oligonucleotide primers (e.g. primer pairs) for amplifying a plurality of (e.g. 5 or more, 8 or more 10 or more, 12 or more 15 or more, 20 or more, 30 or more, 40 or more, 50 or more or substantially all of, or all of): LF, TGFB1, CASP3, TIRAP, CXCL5, TLR2, SAA3, NOS2, CXCL8, AKT1, IRF3, C3, TNFR2, FOS, CXCL3, CCL5, CCR7, LY96, TRAF6, MYD88, STAT2, TLR4, THFRSF1A, IL3RA, CASP8, MAPK8, IRAK1, S100A12, BCL-X1, CCL20, MOD2, LPO, RELA, TNFα, LYZ1, BAX, IL6, CD40, WNT4, IL1-A, CD68, 4MP1, IRAK4, FAS, CD14, S100A9, BCL2, CYP1B1, TNFRSF1A, LBP, NOD2, MX1 and MX2. In particular, the kit may comprise a plurality of (e.g. 5 or more, 8 or more 10 or more, 12 or more 15 or more, 20 or more, 30 or more, 40 or more, 50 or more or substantially all of, or all of primers and/or primer pairs set forth in Supplementary Table 1. The primers may be suitable for and may be provided together with reagents (e.g. reverse transcriptase) suitable for performing RT-qPCR.

In particular, the kit may contain probes for detecting 5 or more, 8 or more 10 or more, 12 or more 15 or more, 20 or more, 30 or more, 40 or more, 50 or more or substantially all of, or all of the gene expression products of: LF, TGFB1, CASP3, TIRAP, CXCL5, TLR2, SAA3, NOS2, CXCL8, AKT1, IRF3, C3, TNFR2, FOS, CXCL3, CCL5, CCR7, LY96, TRAF6, MYD88, STAT2, TLR4, THFRSF1A, IL13RA, CASP8, MAPK8, IRAK1, S100A12, BCL-X1, CCL20, MOD2, LPO, RELA, TNF, LYZ1, BAX, IL6, CD40, WNT4, IL1-A, CD68, MMP1, IRAK4, FAS, CD14, S100A9, BCL2, CYP1B1, TNFRSF1A, LBP, NOD2, MX1, MX2. For example, the kit may contain nucleic acid probes which specifically bind to the mRNA expression products of the genes of interest.

The device can quantify the gene expression level of the genes of interest.

The binding agents may be immobilised on one or more solid supports, for example on a microarray chip.

In addition, the kit may comprise one or more binding agents capable of binding specifically to an expression product of a control gene for example, one for which the expression level is not altered upon stimulation with C. difficile or a C. difficile specific antigen.

The level of expression from this control gene may be measured in order to assist in quantification of the expression products of the genes of interest, and/or for quality assurance of an assay performed using the kit. Preferably a control gene is chosen which is constitutively expressed in the cells of the biological sample (i.e. always expressed, at substantially the same level, under substantially all conditions). Such genes are often referred to as “housekeeping” genes.

The kit may comprise further binding agents capable of binding to expression products of other biomarker genes or control genes. However, in preferred embodiments, the kit comprises binding agents for expression products of less than 1000 different genes, e.g. less than 500 different genes, less than 400, less than 300, less than 250, less than 200, less than 100, or fewer than 70 different genes. For example, the kit may comprise comprises binding agents for expression products of the genes of interest and no more than 10, 20, 30, 40, 50, 60, 70, 80, 90, 100, 150, 200, 250, 300, 400, 500, 600, 700, 800 or 900 additional genes expression products.

The kit is suitable for use in the methods of the invention described in this specification, and may comprise instructions for performing one or more methods of the invention.

### FIGURES

FIG. 1: Determination of the specific IgA content in milk-Classification of the animals (n=9), according to their immune response to the C. diff. vaccine, into the low (n=4) and high (n=5) responder group. The specific IgA content in milk was measured using a sandwich ELISA, the threshold to distinguish between low and high responder animals was set to 8 μg/ml specific IgA.

FIG. 2: Light microscopy of pbMEC isolated from fresh milk immunostained against cytokeratin (the insert shows the negative control, magnification ×200). pbMEC were cytokeratin positive and showed the characteristic cobblestone-like morphology of the cells.

FIG. 3: SOTA analysis revealed an, early (16 genes) (A), intermediate (13 genes) (B) and late (22 genes) (C) expression profile of genes stimulated with C. diff. in the high responder group. Sample fold changes compared to the mean fold change of the whole group are highlighted in the SOTA dendrogram (left) as well as in the SOTA expression profile (right). Fold changes which were higher compared to the mean fold change were highlighted in green, whereas fold changes below the mean fold change were highlighted in red.

FIG. 4: Differences in the gene expression of immediate early expressed genes (19 genes) between the high (B) and low (C) responder group. The SOTA dendrogram (A) as well as the expression profile for the high responder (B) and low responder (C) animals indicated the differential gene expression within both groups.

FIG. 5: Differences in the gene expression of immediate early and intermediate early expressed genes (11 genes) in the high (B) and low (C) responder group. Throughout the SOTA dendrogram (A) as well as the expression profiles for the high (B) and low (C) responder group, indicated a different gene expression pattern within the high and low responder group.

FIG. 6: Differences in the gene expression of late induced genes (21 genes) in the high (B) and low (C) responder group. The SOTA dendrogram (A) as well as the expression profiles for the high (B) and low (C) responder group, indicated a different gene expression pattern within the high and low responder group.

### EXAMPLES

### Methods

### Immunization of the Cows

The animal trail was approved by the government of Upper Bavaria (AZ. 55.2-1-54-71 2532.6-17-2012). Nine healthy Brown Swiss cows in their first lactation were immunized according to a strict immunization scheme 16 times during 31 weeks with a vaccine against Clostridium difficile (C. diff.) (IDT Biologika GmbH, Dessau-Rosslau, Germany). The animals were routinely monitored concerning their health status. Before and one day after each vaccination a general health check was conducted by a veterinarian. The milk of each udder quarter was tested for any bacterial infection or contamination (Tiergesundheitsdienst Bayern e.V., Grub, Germany) before vaccination took place in order to detect any subacute or acute mastitis. The somatic cell count and milk ingredients were analyzed weekly by the Milchprtfring Bayern e.V. (MPR, Wolnzach, Germany). During the whole vaccination period, an average somatic cell count of 63.000 cells/ml±7075.63 cells/mi (n=279) was determined. During the entire experimental phase, two cows showed symptoms of subclinical mastitis and only one cow had an acute mastitis during this time period of 31 weeks. No pbMEC were sampled from the diseased animals, as only pbMEC of healthy cows, were favoured for the experiments. Furthermore, the stool of the animals was tested for C. diff. prior to the immunization. All animals were C. diff. negative (Leiden University, Medical Center).

IgA Against Clostridium difficile—ELISA

For the detection of C. diff. specific IgA in cow milk, a sandwich ELISA was applied. In brief, multiwell plates (96-well, Maxisorp, Nunc®, Sigma-Aldrich, Saint Louis, USA) were coated with C. diff. cells (2.0*10⁸ cells/ml, IDT Biologika GmbH) in coating buffer (50 mM NaHCO₃, pH 9.6, Merck Chemicals GmbH, Darmstadt Germany) for 2 h at 70° C. and afterwards overnight at 4° C. The coating was terminated by the 9 addition of 200 μl blocking buffer (2% gelatin, Sigma-Aldrich, in PBST, 1 h, 37° C.).

The ELISA plate was washed four times with phosphate buffered saline-Tween buffer (1 g/l Tween 20, Merck Chemicals GmbH, PBST). The C. diff. specific IgA standard was prepared in dilution buffer (0.2% gelatin, Sigma-Aldrich, in PBST, 62.5 ng/ml-4*10³ ng/μl). The skim milk samples were diluted 1:10 with dilution buffer. Standard dilutions, samples and intra-assay controls were applied in duplicates to the pre-coated plate (1.5 h, 37° C.). The ELISA plate was washed (4×) and the HRP conjugated sheep anti-bovine IgA (Bethyl Laboratories, Inc., Montgomery, USA, 1:70,000) was added to each well (1.5 h, 37° C., light protected). Afterwards, the ELISA plate was washed (4×) and the HRP substrate [15] was added to the wells to induce the substrate reaction. After 40 min the substrate reaction was stopped by the addition of 2M H₂SO₄. Extinction was measured after 30 min at 450 nm using a microplate reader (Sunrise™, Tecan Group Ltd., Mannedorf, Switzerland). The amount of C. diff. specific IgA was determined based on the standard curve (Magellan™ V6.6 software).

3D Cell Culture of pbMEC

The pbMEC were isolated from fresh milk of 9 healthy Brown Swiss cows in mid-lactation, as described in Sorg et al. (2013a) and Danowski et al. (2013) [16,17]. In brief, fresh milk was defatted (10 min, 1850×g), the remaining cell pellet was washed several times with 1× Hanks Balanced Salt solution (Sigma-Aldrich), that further contained antibiotics and antimycotics [16,17]. The remaining cell suspension was filtered twice (EASYstrainer™: 40 μm; 100 μm, Greiner Bio-One GmbH, Frickenhausen, Germany) to remove lipid droplets and cell aggregates. The pbMEC were afterwards resuspended in DMEM/F-12 Ham solution supplemented with Penicillin/Streptomycin, Amphotericin B, ITS liquid media supplement (Sigma-Aldrich) and FBS (Gibco® Lifetechnologies GmbH, Darmstadt, Germany) and cultured (37° C., 5% CO₂) in 3D cell culture on 6-Well plates, coated with 2.4 mg/ml Matrigel® (Corning Inc., Corning, N.Y., USA), until confluency. pbMEC were sub-cultivated using 0.25% Trypsin-EDTA solution (Sigma-Aldrich). After the second passage the cells were detached with 0.25% Trypsin-EDTA solution and prepared for cryopreservation. The cells were counted using the TC10™ Automated Cell Counter (Bio-Rad Laboratories GmbH, Munich, Germany), 1*10⁵-5*10⁵ cells were resuspended in cryopreservation medium containing 70% DMEM F12-Ham, 20% FBS and 10% DMSO (Sigma-Aldrich) and stored in liquid nitrogen, until pbMEC from all animals had been sampled. For the experimental set-up, pbMEC were thawed and reseeded at 2*10⁴ cells per well of a 6-well plate (Greiner Bio-One GmbH), coated with 2.4 mg/ml Matrigel®, for the immune stimulatory experiments or 1*10⁴ cells per chamber of a 8-well LabTec chamber slide (LAB-Tek, Nunc, GmbH, Langenselbold, Germany) for immunocytochemistry (IC).

Immune Stimulation of pbMEC with Formalin Inactivated Clostridium difficile

In order to calculate the multiplicity of infection (MOI) per cultured cell, three wells per animal served as counting wells. Therefore the cells, upon reaching confluency, were detached using 0.25% Trypsin-EDTA solution and counted in the TC10” Automated Cell Counter (Bio-Rad Laboratories GmbH, Munich, Germany), using life-dead staining with 0.4% trypan blue (Bio-Rad Laboratories GmbH). The mean value of the counted and living cells served as the estimate cell count for all other cells of the animal within the experiment. Cell culture replicates of pbMEC were then induced with formalin inactivated C. diff. (IDT Biologika GmbH) with a MOI of 70 colony forming units per cultured cell. The MOI was chosen upon findings in preliminary experiments. A higher MOI was chosen, compared to the literature, as it is already known that gram-positive pathogens induce only a weak immune response in pbMEC [12,18,19]. To target the immediate as well as the intermediate and late immune response, pbMEC were treated with the C. diff. for 6 h, 24 h and 72 h respectively. In order to obtain representative data, control wells with untreated pbMEC were also sampled in biological triplicates at each time-point (0 h, 6 h, 24 h, and 72 h). To avoid side effects of antibiotics, antimycotics and FBS, the cells were supplemented with DMEM/F-12 Ham medium, supplemented only with ITS, 48 h pre-infection. This so called infection medium was refreshed immediately before treatment begin. After the treatment, pbMEC were washed with phosphate buffered saline (PBS) and further lysed in Qiazol (Qiagen, Hilden, Germany) of the miRNeasy Micro Kit (Qiagen).

### Mycoplasma Test

In order to detect the presence of contaminant mycoplasma species in cell culture, the PCR Mycoplasma Test Kit (AppliChem GmbH, Darmstadt, Germany) was used according to the manufacturer's protocol. Cell culture supernatants were sampled for each animal and stored at 80° C. until further processing.

### Immunocytochemistry

pbMEC were cultured on 8-well LabTec chamber Slides (LAB-Tec, Nunc, GmbH, Langenselbold, Germany) to confirm the epithelial character of the cells cultured in 3D cell culture with immunocytochemistry. The IC was conducted as described earlier in Sorg et al. (2013a) and Danowski et al. (2013) [12,16,17]. For the cytokeratin staining, the monoclonal mouse anti-cytokeratin pan antibody clone C-11 165 (1:400 in PBST, Sigma-Aldrich) was used.

### RNA Extraction and Reverse Transcription

RNA was extracted according to the manufacturer's protocol of the miRNeasy Micro Kit (Qiagen), with slight adaptions. The miRNeasy Micro spin column was incubated for 5 min with buffer RPE after the second addition of buffer RPE, to reduce contaminations of the RNA with guanidine thiocyanate. The RNA concentration was evaluated using the Nanodrop ND-1000 spectrophotometer (Peqlab, Erlangen, Germany). The RNA integrity was analyzed with the 2100 Bioanalyzer on the 6000 nano chips and the RNA 6000 nano Kit (Agilent Technologies, Waldbronn, Germany) according to the manufacturer's instructions. RNA was stored at −80° C. until further analysis. For reverse transcription of RNA to cDNA, 400 ng of RNA were mixed together with a master mix containing 5× buffer, 0.5 mM dNTPs, 0.5M Oligo-d(T) primers (Fermentas, St Leon-Rot, Germany), 2.5 μM random hexamer primers (Invitrogen Life Technologies, Darmstadt, Germany) and 100 U M-MLV H(−) reverse transcriptase (Promega, Mannheim, Germany) in a total volume of 20 μl. After the reverse transcription, the cDNA was diluted 1:1 to and end volume of 40 μl. RNA isolated from bovine mammary gland tissue and bovine spleen tissue was used as a positive control. Furthermore a non-template control (NTC) was carried along within each 96-well plate (4titude, Wotton, Great Britain) to screen for contaminations of the reaction mixture. The RT-PCR reactions were conducted in 96-well plates on the T-Personal Thermocycler (Biometra, Göttingen, Germany)(Annealing: 21° C., 10 min, transcription phase: 48° C., 50 min, degrading phase: 90° C., 2 min). The remaining cDNA was stored at 20° C.

### RT-qPCR Primer Design

Bovine specific primer pairs were designed using published bovine nucleic acid sequences of the National Center for Biotechnology Information gene database (NCBI, National Library of Medicine, Bethesda Md., USA). 68 bovine specific primer pairs were generated (Sigma-Aldrich), among them were 7 primer pairs for the reference genes GAPDH, YWHAZ, H3F3A, ACTy1, 18srRNA, Cyt8, UBB and respectively 61 primer pairs for target genes coding for proteins involved in inflammatory pathways (Supplementary Table 1). Primers were designed using Primer3web version 4.0.0 [20,21]. All primers were tested concerning their specificity and performance. All primers had their optimal annealing temperature (TM) at 60° C. Each designed assay was tested within cDNA generated from udder parenchyma tissue, spleen tissue and pbMEC to be sure that the genes were expressed in the particular tissue. Furthermore, each qPCR assay was tested for its amplification efficiency according to the minimum information for the publication of quantitative real-time PCR experiments (MIQE) guidelines [22]. Only assays with a PCR efficiency >85% were used within RT-qPCR quantification. The so optimized assays were ready for subsequent RT-qPCR experiments.

### RT-qPCR Measurements

The RT-qPCR measurements were conducted on the BioMark™ HD 96×96 system (Fluidigm, San Francisco, Calif., USA) as described in Sorg, et al. (2013) with slight optimizations [12]. The cDNA was specifically pre-amplified for 16 cycles using 67 primer pairs. The 18srRNA primer pair was excluded from pre-amplification as it was scored as highly expressed gene. In brief 2 μl of cDNA (10ng/μl) were pre-amplified in a total volume of 15 μl with a final primer concentration of 25 nM using the iQ Supermix (Bio-Rad) according to the following temperature protocol: Activation of polymerase 95° C. for 3 min, 16 cycles of denaturation at 95° C. for 15 sec and 4 min of annealing and extension at 59° C. The cDNA was diluted 20 times after the pre-amplification reaction and stored at 20° C. until further analysis. For the determination of the Cq values, 4 BioMark” 96×96 Gene expression (GE) Dynamic Array chips (Fluidigm) were used. The efficiency of all primer assays was tested on the first 96×96 GE dynamic array (Fluidigm). Furthermore each 96×96 GE dynamic array contained positive controls, one no transcription control (NTC) and one control sample to test for possible genomic contaminations, called ValidPrime® (TATAA Biocenter, Gothenburg, Sweden). ValidPrime® was a good alternative to avoid no reverse transcriptase controls in reverse transcription PCR, as it tests all samples for the presence of genomic DNA during the RT-qPCR run. Two stably expressed samples of the first 96×96 GE dynamic array were chosen as between-chip calibrators, and hence were measured on all 4 chips. For the sample premix, 2.5 μl SsoFast” EvaGreen supermix (Bio-Rad), 0.1 μl of ROX (4× diluted, Invitrogen), 0.25 μl of 20× binding dye loading reagent (Fluidigm), 1 μl pre-amplified and 1:20 diluted cDNA and 1.15 μl water were combined to a final volume of 51. The 5 μl assay mix consisted of 2.5 μl of 5 μM primer pairs (final concentration of primers in an individual reaction: 250 nM) and 2.5 μl of 2×GE assay loading reagent (Fluidigm). The sample and assay pre-mix were transferred to the primed 96×96 GE dynamic array, where they were automatically mixed inside the chip with the Fluidigm® IFC controller. The RT-qPCR was conducted with the following protocol on the BioMark™ system: 98° C., 40 sec followed by 30 cycles of 95° C. for 10 sec and 60° C. for 40 sec, followed by a melting curve analysis to reveal the specificity of the primer pairs. The Fluidigm Real-Time PCR Analysis Software Version 4.1.2 237 (Fluidigm) was used for data handling and analysis. The RT-qPCR reactions described here were performed according to the minimum information for the publication of quantitative real-time PCR experiments (MIQE) guidelines [22].

### Data Pre-Processing and Data Analysis

The qPCR reactions were validated with the Fluidigm Real-Time PCR Analysis software Version 4.1.2 (Fluidigm). Primer pairs with too many missing data were excluded from further analysis (CYP1A1, IL1-B, IL10, CASP1, HP, TAP, LAP, CCL2). Furthermore, standard curves generated on the first BioMark® 96×96 GE Dynamic Array chip (Fluidigm), were used to determine the efficiency of the primer pairs and the cut-off value for the gene expression data. The dynamic range of the primer assays was tested and the cut-off value was therefore set to 26. The raw data was pre-processed in GenEx Enterprise Version 6 (MultiD Analyses AB, Gothenburg, Sweden). Within GenEx values larger than 26 were treated as missing data, the cut-off was set to a Cq-value of 26, and missing data was treated with an offset of “+1”. Furthermore the genomic background in each sample was evaluated and an inter-plate calibration was conducted using the mean value of the two inter-plate calibrator samples. The so pre-processed Cq values were normalized with a set of 7 reference genes, as suggested by the ‘Normfinder’ tool within GenEx (MultiD Analyses AB). Additionally the normalized Cq values were further normalized to the corresponding reference samples, which were represented by the Cq values of untreated control wells, which were sampled at treatment start (time-point 0 h). The fold changes (2\hat{ }^{(-ΔΔCq)}) were calculated as described in Livak and Schmittgen (2001) [23]. The statistical analysis of the data was conducted with SigmaPlot 12.0 (Systat). Before p-values were calculated, the normal distribution of the data sets was proofed using the Shapiro Wilk normality test. For not normally distributed data, a signed rank test was conducted. To evaluate the treatment effect of C. diff. the delta-Cq values of the treated groups were compared to the untreated control groups, using a paired t-test. Significant differences in the gene expression between the different treatment time-points (6 h vs. 24 h, 6 h vs. 72 h, 24 h vs. 72 h) were also evaluated using a paired t-test. Furthermore a normal t-test was conducted in order to find differentially expressed genes between the high and low responder group. Gene expression changes with p-values between 0.1 and 0.05 were considered as distinct changes in gene expression, whereas p-values below 0.05 were considered as statistically significant changes in gene expression (*p≤0.05, ** p≤0.01, *** p≤0.001). As no correction for multiple testing was imposed on the p-values, this study has to be considered as explorative study. For the identification of similar gene expression profiles, a cluster analysis with the self-organizing tree algorithm (SOTA) was conducted with the Multi Experiment Viewer software (MeV 4.9.0, TM4) [24].

### Results

C. Diff. Specific IgA in Cow Milk

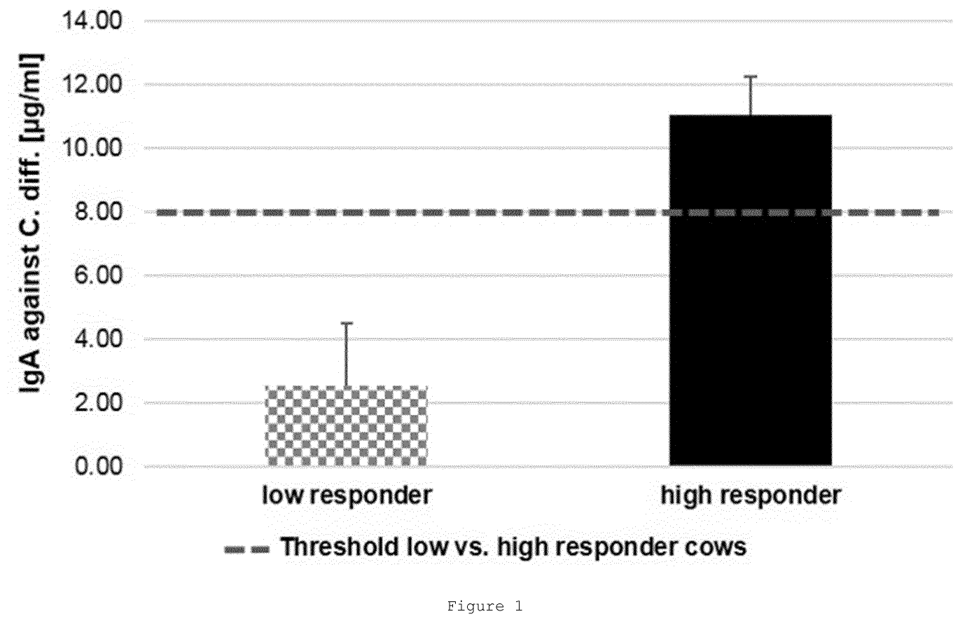

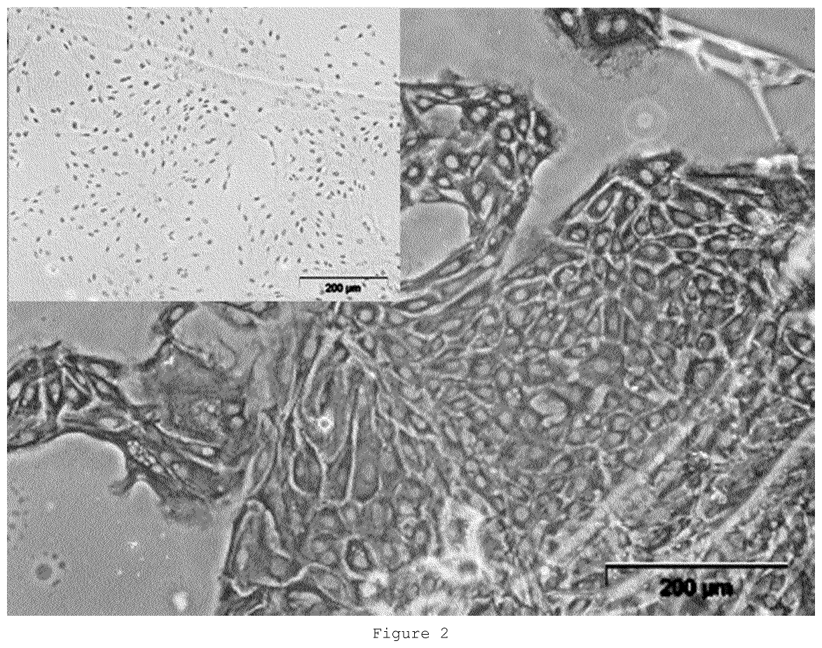

The IgA content in milk was determined using an IgA ELISA as described above. In order to distinguish between high and low responder cows, the threshold of C. diff. specific IgA in secreted milk of the immunized animals, was set to 8 μg/ml milk. Therefore 4 animals were termed as low responder animals with an average specific IgA content of 2.6 μg/ml±1.9 μg/ml and 5 animals were termed as high responder animals with an average specific IgA content of 11.1 μg/ml±1.2 μg/ml milk (p≤0.001) (FIG. 1).

pbMEC Cell Culture—IC and Mycoplasma Test

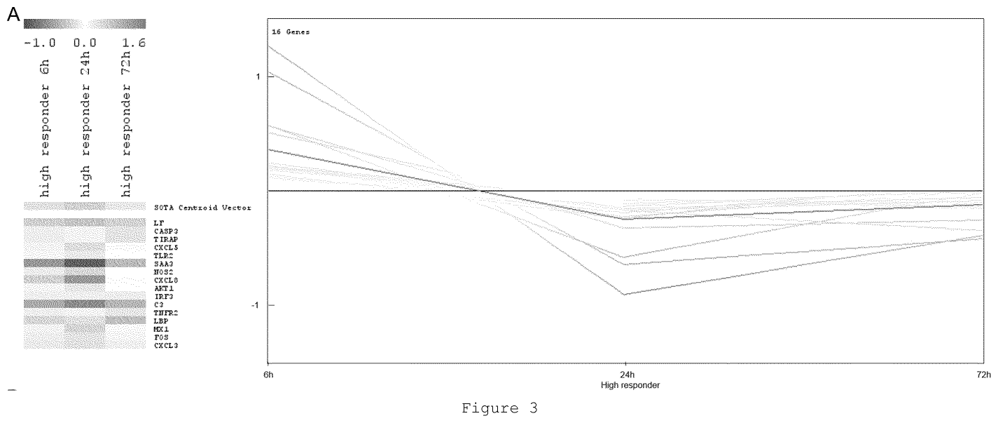

In order to confirm the epithelial character of the pbMEC cultured in 3D cell culture, an IC was conducted. All cells used within the cell culture were cytokeratin positive and showed the typical cobblestone-like morphology, which is characteristic for pbMEC (FIG. 2). Therefore, cross-contaminations with other cells could be excluded. Furthermore, it could be shown that the cells of all animals were mycoplasma free (PCR Mycoplasma Test Kit, AppliChem GmbH, Darmstadt, Germany).

### Quality Control of the Extracted RNA and the RT-qPCR Assays

The quality of the extracted RNA was proofed as described before. In brief, the total RNA yield was determined on the Nanodrop ND-1000 spectrophotometer (Peqlab). An overall RNA yield of 335.21 ng/μl±15.00 ng/μl could be determined (n=314). The RNA integrity which was analyzed with the 2100 Bioanalyzer on the 6000 Nano chips was measured for 70 RNA samples that were randomly collected over all 4 time-points. An average RIN value of 9.94±0.13 (n=70) could be determined, indicating a very good integrity of all RNA samples, as the highest value to achieve is a RIN of 10. Furthermore, the designed qPCR assays were tested for their efficiency in qPCR reactions according to the MIQE guidelines [22], using standard curves of serial diluted sample material. The performance of the assays was tested on the BioMark” 96×96 GE dynamic array, assays with a bad PCR amplification efficiency were excluded from further analysis (CYP1A1, IL1-B, IL10, CASP1, HP, TAP, LAP, CCL2). The analysis of the remaining 60 qPCR assays resulted in an average r2-value of 0.98±0.003 (n=60) and an average PCR efficiency of 1.08±0.016 (n=60).

Effect of the C. Diff. Treatment on the Gene Expression within the High Responder Cows

According to the immunoglobulin yield obtained in the milk, the vaccinated cows showed a quite diverse immune response. We therefore applied a gene expression profiling method, in order to find molecular biomarkers in innate immunity. The intention was to bring those molecular biomarkers into account with animals that show a fast and efficient immune response. A detailed listing of the fold changes in gene expression and the calculated p-values, that were determined in a paired t-test for the treatment effect and the time effect, can be found in Supplementary Table 2. For the identification of differences within the expression profiles of low and high responder animals, a cluster analysis with the self-organizing tree algorithm (SOTA) was conducted applying the Multi Experiment Viewer software (MeV 4.9.0, TM4) [24]. The analysis was done based on mean centered fold change values. Genes with higher fold changes than the mean are highlighted in green, whereas fold changes below the mean fold change are highlighted in red. With this method, the time course of gene expression changes within the high and low responder groups could be illustrated whereby the genes were clustered concerning their early, intermediate or late gene expression.

### SOTA Analysis of RT-qPCR Data

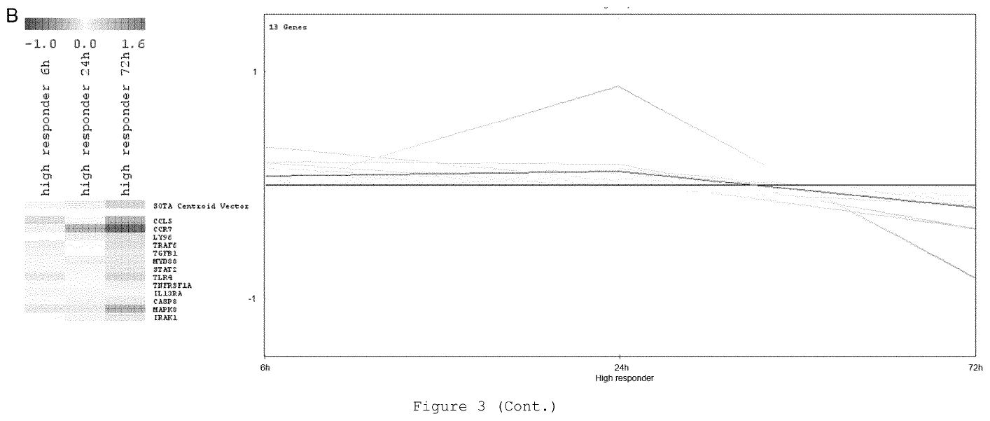

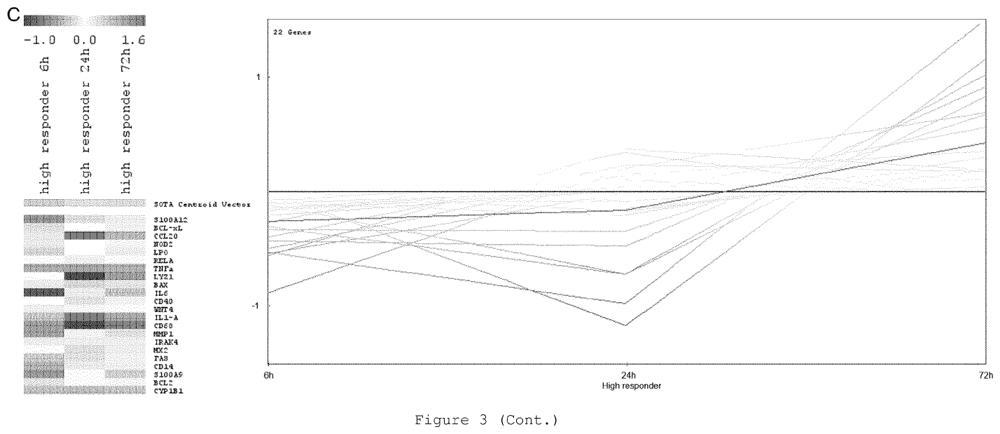

Within the SOTA analysis of the high and the low responder group, the genes coding for FcRn and pIGR were not taken into account, as they didn't contribute to the scientific question. The SOTA analysis for the high responder group revealed three clusters, one for the early induced genes (FIG. 3A), one for the intermediate (FIG. 3B) induced genes and one for the genes which were mostly induced after 72 h (FIG. 3C) of the immune treatment.

The first cluster contained 16 genes, which were early induced after immune stimulation. Among them were some really strong induced genes coding for the acute phase protein SAA3, the antimicrobial peptide LF, the complement component C3, components of the TLR pathway like LBP, TLR2 and TIRAP and the chemokines CXCL5, CXCL3 and CXCL8 (FIG. 3A). The treatment effect with C. diff. was statistically evaluated using a paired t-test (Supplementary Table 2). A differential up-regulation in gene expression due to the immune stimulation could be determined for CXCL8, CXCL3 and TIRAP. The time dependent effect on the gene expression, that is shown in supplementary table S2, could be seen in the SOTA dendrogram as well (FIG. 3A).

Within the second cluster, 13 genes could be detected, which were rather early (6 h) and intermediately (24 h) induced (FIG. 3B). Genes coding for chemokines like CCL5, CCR7, IL13RA, for components of the TLR-pathway, like TLR4, LY96, MYD88, TRAF6 and IRAK1 and the gene coding for MAPK8 were strongly induced either early or intermediately. According to a paired t-test, the gene expression of genes coding for IRAK1 and TRAF6 were differentially up-regulated due to the immune stimulation (FIG. 3B and Supplementary Table 1). The regulation of the gene expression due to different treatment time-points in this cluster could be directly seen within the SOTA dendrogram and the expression graph (FIG. 3) and was verified with a paired t-test (Supplementary Table 2).

Within the third cluster, 22 intermediate to lately induced genes could be detected. Among them were strongly induced genes coding for TNFα, CD68, CD14 and CYP1B1. Furthermore the genes coding for the so called danger associated molecular pattern molecules S100A9 and S100A12, for the antimicrobial peptides LYZ1 and LPO, for the chemokines and inflammatory cytokines CCL20, IL6, IL1-A and for components of the apoptotic pathway, like FAS, the scavenger receptor CD68 and the gene coding for MMP1, were differentially induced due to the stimulation with C. diff. (Supplementary Table 2). This trend was also visible in the SOTA dendrogram and expression profile (FIG. 3C), where again the temporal regulation of the gene expression of the immune genes could be clearly seen (FIG. 3C; Supplementary Table 2).

### Comparison of the Gene Expression Pattern of Low and High Responder Cows During Different Treatment Time-Points

The direct comparison of the gene expression pattern of high and low responder cows indicated a distinct higher induction of the gene expression within the high responder group during all three time-points.

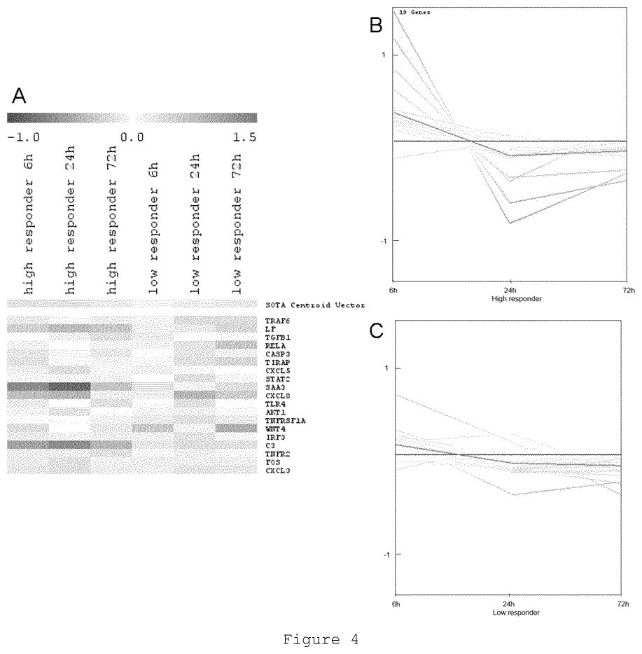

The genes clustered together concerning their induction time within the immune response. The first cluster consisted of 19 genes, which belonged to the genes which were early expressed. Within the SOTA dendrogram (FIG. 4A), it could be detected, that those genes were induced through the C. diff. stimulation in the high responder group as well as in the low responder group, even if the gene expression within the low responder group was partly lower (FIG. 4A, B, C). The potential lower induction of gene expression in the low responder group could be clearly seen within the SOTA expression graphs (FIG. 4B, C).

Most of the early induced genes within the high responder group, showed a more distinct up-regulation after 6 h of immune stimulation and a strong down-regulation after 24 h. In the low responder group however, only a few of those 19 genes, showed a higher fold change compared to the mean fold change of the whole group, and the gene expression within the low responder group declined after 24 h. Among the strongly induced genes in the high responder group, were genes coding for the antimicrobial peptide LF, chemokines like CXCL8, CXCL5, CXCL3 as well as the gene coding for the acute phase protein SAA3 and the complement component C3. Furthermore, genes coding for components of the TLR-pathway, like TIRAP, TRAF6 and RELA were differentially induced in the high responder group when compared to the low responder group in a normal t-test (Table 1; FIG. 4 and Supplementary Table 2).

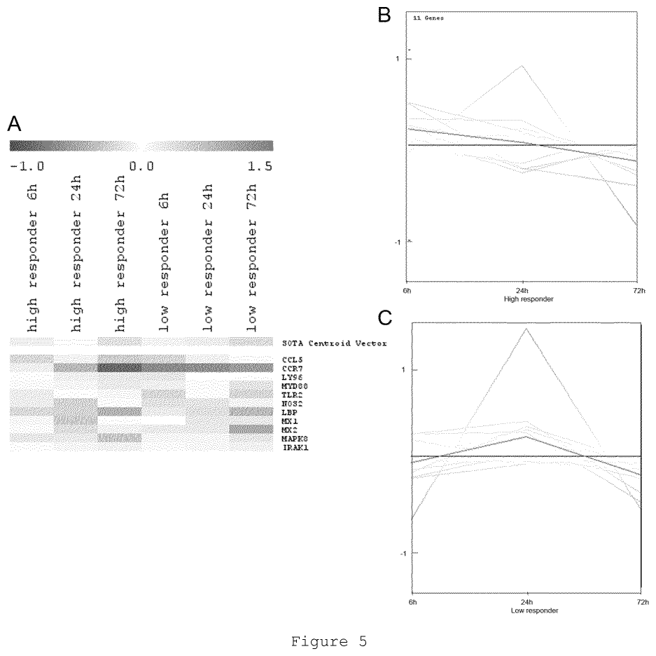

Eleven genes clustered together in the second cluster. Those genes were rather early or intermediately induced. The dendrogram, as well as the expression profile (FIG. 5 A, B), clearly indicated that the high responder group tended to up-regulate the genes within this cluster already after 6 h, despite of the gene coding for the chemokine receptor CCR7 (FIG. 5 A, B). This accounts for an early as well as prolonged induction of the gene expression of genes coding for important chemokines like CCL5 and for components of the TLR-pathway, like LY96, MYD88, TLR2 and IRAK1. Most of those genes however were down-regulated 6 h post-immune stimulation within the low responder group, and were only induced after 24 h. Some genes like the gene coding for NOS2, LBP, MX1 and MX2 were down-regulated within the high responder group after 24 h whereas their gene expression was induced after this period of time within the low responder group, indicating a contrary gene regulation between both groups. By contrast the genes coding for CCL5, IRAK1 and MAPK8 showed exactly the contrary, as they were up-regulated within the high responder group, but down-regulated within the low responder group. Using a normal t-test (Table 1) a distinct and differential up-regulation in the gene expression between the high and low responder group could be revealed for LY96, MYD88, IRAK1, CCL5 and MAPK8 (Table 1; FIG. 5; Supplementary Table 2).

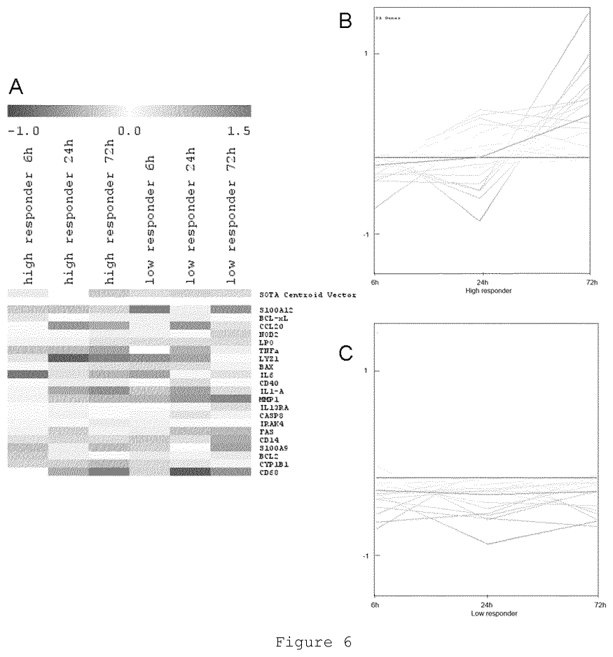

The third cluster consisted of 21 genes, which belonged to the genes which were rather late induced after 72 h and partly after 24 h of immune stimulation. The SOTA dendrogram revealed that those genes were especially induced in the high responder group after 72 h, when the color coding is compared with the low responder group at the same time-point (FIG. 6A). It could also be seen that those genes were not earlier induced within the low responder group. The pbMEC in the low responder group hardly showed any induction of the gene expression of those immune genes after the immune stimulation with C. diff. (Table 1). Differentially up-regulated genes among the late expressed genes were some late chemokines and inflammatory cytokines, like IL6, IL1-A and IL13RA, genes coding for the danger associated molecular pattern molecules S100A9 and S100A12, for the antimicrobial peptides LYZ1 and LPO, components of the TLR pathway, like CD14, genes coding for the pro-apoptotic factors FAS, CASP8 and BAX and genes coding for CD68, CD40, MMP1 and NOD2. The expression graphs visualize the up- and down-regulation in gene expression over-time (Table 1; FIG. 6).

### Discussion

We investigated the gene expression profile of pbMEC extracted from milk of immunized cows concerning differences in the molecular gene expression patterns between high and low responder cows, after treatment with a bacterial C. diff. stimuli. High responder cows (n=5) were classified in accordance to their high specific IgA amounts in milk and fast immune response, whereas low responder cows (n=4) showed less antibody production after repeated immunization. The aim was to establish a defined gene expression pattern or a special set of genes of chemokines, immune receptors, acute phase proteins and more which could serve as molecular biomarkers for the pre-selection of cows, before the animals are immunized for the immune milk production.

We chose the pbMEC as cell culture model for our infection studies, as it is known and already proven, that bovine mammary epithelial cells have important tasks within the bovine mammary gland [11]. The rather low changes in the gene expression levels were expected, as it has already been shown by Strandberg et al. (2005), Griesbeck-Zilch et al. (2008) and Sorg et al. (2013) that gram-positive pathogens provoke only a weak innate immune response [12],[13],[14]. We chose the gram-positive pathogen C. diff., as it causes severe CDAD in immune suppressed and elderly humans [3]. We think that the treatment of CDAD with immune milk has great advantages, like the maintenance of the healthy commensal gut microbiota and the prevention of the formation of resistant bacteria due to the use of natural polyclonal animal derived antibodies. The pathogen specific polyclonal IgA should specifically neutralization C. diff. bacteria, so that the relapse rate and of course antibiotic treatments could be reduced to a minimum.

### TLR Pathway

It has been shown from Strandberg et al. (2005) that the innate host defence of pbMEC depends on germline-encoded receptors that recognize conserved structures expressed by a wide variety of microbes [13]. Hence it is known that they express Toll-like receptors on their cell surface [18]. Therefore, pbMEC should be able to recognize the gram-positive pathogen C. diff. upon the recognition of the bacterial cell wall component lipoteichoic acid (LTA) through the pattern recognition receptors CD14, TLR2 and TLR4 [25]. It has been shown within our study that the gene expression of the two receptors TLR2 and TLR4 was mainly unaffected within the low and high responder group by the bacterial stimuli. This is in accordance with a study of Strandberg et al. (2005) [13]. They postulated that pbMEC contain a fully functional and constitutively active TLR signaling pathway, that is immediately responsive to a bacterial challenge, so that not the gene expression of the receptors itself is responsible for the inefficient activation of NFkB and hence transcription of cytokines, but deficits in the downstream signaling [13]. However a distinct and statistically significant higher gene expression within the high responder group, compared to the low responder group, could be detected for CD14, which is in accordance with the findings of Lutzow et al. (2008) [26].

In order to investigate whether any differences in the activation and downstream signaling cascade were present through the treatment with C. diff. and between the high and low responder group, we evaluated the gene expression profiles of LY96, LBP, CD14, MYD88, TIRAP, TRAF6, IRAK4, IRAK1 and RELA. We found significant as well as distinct changes in the gene expression profile of genes coding for LY96, CD14, TIRAP, IRAK1 and RELA especially within the high responder group. Furthermore a significantly as well as distinctly higher gene expression could be detected in this group compared to the low responder group for MYD88, TRAF6, LY96, CD14, TIRAP and RELA. Therefore we agree with Strandberg et al. (2005) [13] in the finding, that deficits in the downstream signaling process of the low responder animals are responsible for the relatively low expression of RELA, which is also known as the NF-476 kappa-B p65 subunit.

### Chemokine Activation

It is known that upon activation, NFkB translocates into the nucleus and initiates the gene expression of a variety of pro-inflammatory effector genes, like chemokines and cytokines, survival and proliferation associated genes [27]. Targets also include adhesion molecules, acute phase proteins like SAA-proteins and inducible enzymes [27,28]. We could observe this effect when the gene expression of prominent chemokines of the high responder group was compared to the low responder group. The gene expression of CXCL8, CCL5, CXCL5, IL6, IL1-A and IL13RA was distinctly higher induced within the high responder group. Especially CXCL8, which is known to be one of the major initiators of the inflammatory response has been shown to be essential for the immediate recruitment of leukocytes into the bovine mammary gland and hence is responsible for the elimination of invading pathogens [13,29].

The CXCL8 gene expression was substantially higher within the high responder fibroblasts, after stimulation with LPS in cell culture. Additionally, a study of Griesbeck-Zilch et al. (2008), also showed a significant and early induction of CCL5 gene expression after stimulation of pbMEC with S. aureus [14]. Furthermore, Lahouassa et al. (2007) showed that bMEC are able to produce and release chemokines, even without the up-regulation of the anti-inflammatory cytokine IL10, which we could also not measure within our study [30]. As the genes coding for chemokines and inflammatory cytokines were particularly stronger induced within the high responder group, it could be possible that the high responder group is more efficient in the initiation of the inflammatory reaction and the recruitment of other immune components to the site of infection.

### Gene Expression Pattern of Antimicrobial Peptides

The activation of the transcription factor NFκB is also known to induce the gene expression and production of antimicrobial peptides like lysozyme 1 (LYZ1), lacto-peroxidase (LPO) and lactoferrin (LF). Normally, antimicrobial peptides are constitutively expressed, even if no direct bacterial stimuli is present. Those peptides are mostly built in cells which are permanently exposed to bacteria, like epithelial cells. Lactoferrin for example shows a bacteriostatic effect through its capability to bind iron, which is essential for bacterial growth [17], however in contrast to Griesbeck-Zilch et al. (2008) no up-regulation of the gene expression of LF could be detected in both treatment groups [14]. Lysozyme is also a bactericidal protein that cleaves peptidoglycans of the cell wall of gram-positive and gram-negative bacteria. The third antimicrobial peptide analyzed in this study was the lacto-peroxidase, which is able to kill or inhibit bacteria in the presence of thiocyanate and hydrogen peroxide [31]. In our study only a significant induction of the gene expression of LPO and LYZ1 could be detected within the high responder group. Furthermore, LPO as well as LYZ1 gene expression were significantly higher within the high responder group when compared to the low responder group.

### Danger Associated Molecular Pattern Molecules

Acute phase proteins like S100A12 and S100A9 also participate in the regulation of inflammatory processes. Furthermore, both acute phase proteins are involved in the induction of cytokine and chemokine production, the significant higher gene expression levels in the high responder group compared to the low responder group could therefore, together with the chemokines, also contribute to a better activation of immune components, resulting in a stronger and faster adaptive immune response than in the low responder group [32,33]. The induction of S100A12 gene expression through gram-positive pathogens has already been shown in Lutzow et 529 al. (2008), Sorg et al. (2013) and Günther et al. (2009), so that the assumption arose that the molecules maybe involved in the initial response to bacterial infection [11,12,26].

### Apoptosis Related Genes

Apoptosis is an important biochemical process, responsible for the proper development and function of the immune system. It has already been shown that apoptosis occurs in response to S. aureus infection in bovine mammary epithelial cell lines and in primary bovine epithelial cells [34]. Considering the induction of apoptosis, the pbMEC of the high responder group, also showed a significant stronger induction of pro-apoptotic genes like Bax, FAS, CASPASE 8 and CASPASE 3 post infection compared to the low responder group. This finding could indicate that the cells of the high responder group were subjected to stronger apoptotic events.

### CONCLUSION

When both gene expression patterns of the TLR signaling pathway and the gene expression of the effector molecules are compared between the low and high responder group, it seems as if the high responder animals are capable to better and even faster induce the innate immune system.

The higher gene expression levels of factors of the TLR-pathway, cytokines and antimicrobial peptides in pbMEC of the high responder group could be advantageous for the recruitment and activation of components of the immune system, resulting in a stronger and faster adaptive immune response than in the low responder group. That in turn leads to a faster induction of antibody producing B-cells and to higher immunoglobulin concentrations in the milk. It might be possible that the gene expression pattern of the pbMEC during infection together with the gene expression pattern of the bovine lymphocytes is the key to new molecular biomarkers, which can be used to identify cows, with an effective immune response and respectively a high amount of immunoglobulins produced in milk.

We are confident that molecular biomarkers will facilitate the pre-selection and hence optimization of the animal health and productivity. So far genes coding for components of the TLR-pathway (LY96, CD14, TIRAP and RELA), the chemokines like CXCL8, CCL5, CXCL5, inflammatory cytokines (IL6, IL1-A), antimicrobial peptides (LYZ1, LPO) and danger associated molecular pattern molecules (S100A9, S100A12) are promising as candidates for molecular markers, as they were differentially expressed between the low and high immunoglobulin responder group.

TABLE 1
Differences in the gene expression of high (n = 5) and low
(n = 4) responder cows evaluated by a normal t-test.

| | Time Point | | |
|---|---|---|---|
| | C. diff. 6 h¹ | C. diff. 24 h¹ | C. diff. 72 h¹ |
| Genes | Low vs. High² | Low vs. High² | Low vs. High² |

TLR pathway

| | | | |
|---|---|---|---|
| LY96 | | *** | ** |
| CD14 | + | *** | * |
| MYD88 | | + | |
| TIRAP | * | ** | *** |
| TRAF6 | | + | |
| IRAK4 | | * | |
| IRAK1 | | + | |
| RELA | | ** | |

Chemokines

| | | | |
|---|---|---|---|
| CCL5 | + | * | |
| CXCL5 | + | | |
| CXCL8 | + | | + |
| IL13RA | | * | + |

Inflammatory cytokines

| | | | |
|---|---|---|---|
| IL1-A | | | + |
| IL6 | ** | + | * |

Acute phase proteins/danger associated molecular pattern molecules

| | |
|---|---|
| S100A9 | * |
| S100A12 | * |

Antimicrobial peptides

| | |
|---|---|
| LYZ1 | ** |
| LPO | * |

Apoptosis

| | | | |
|---|---|---|---|
| FAS | * | ** | * |
| TNFRSF1A | | + | |
| CASP8 | | ** | + |
| CASP3 | * | * | |
| BAX | | ** | |
| BCL-2 | + | | * |

Scavenger Receptor

| | | | |
|---|---|---|---|
| CD68 | ** | * | |
| CD40 | ** | ** | * |

JAK-STAT signaling

| | |
|---|---|
| STAT2 | ** |

MAPK signaling

| | | |
|---|---|---|
| MAPK 8 | ** | * |

Others

| | | |
|---|---|---|
| MMP1 | * | ** |
| MX2 | + | |
| NOD2 | | * |
| AKT1 | * | |
| WNT4 | | + |

¹Treatment time with C. diff. in hours
²low responder animals versus high responder animals
*p ≤ 0.05,
**p ≤ 0.01,
*** p ≤ 0.001
+ distinct changes (0.01 ≤ p < 0.05)

SUPPLEMENTARY TABLE 1
Gene and Primer details for RT-qPCR

| | | | |
|---|---|---|---|
| | NCBI | | |
| | reference | Primer sequence | |
| | sequence | (5′->3′) | L¹ |
| Gene name | number | Forward Reverse | [bp] |

TLR pathway

| | | | |
|---|---|---|---|
| Toll-like | NM_ | CATTCCTGGCAAGTC | 201 |
| receptor | 174197.2 | GATTATCGGAATGGC | |
| 2 (TLR2) | | CTTCTTGTCAATGG | |
| Toll-like | NM_ | TGCTGGCTGCAAAAA | 213 |
| receptor | 174198.6 | GTATGTTACGGCTTT | |
| 4 (TLR4) | | TGTGGAAACC | |
| Lymphocyte | NM_ | TGTTTCAATACGTTC | 300 |
| antigen | 001046517.1 | TGAGCCCTCAGTGTT | |
| 96 (LY96) | | CCCCTCGATGG | |
| Lipopoly- | NM_ | TCCCAGTTGCTTTCC | 194 |
| saccharide | 001038674.2 | TTGCTGCGGAAGGAC | |
| binding | | TTGGTGTTCT | |
| protein | | | |
| (LBP) | | | |
| CD14 | NM_ | GCAGCCTGGAACAGT | 124 |
| molecule | 174008.1 | TTCTCACCAGAAGGT | |
| (CD14) | | GAGCAGGAAC | |
| Myeloid | NM_ | CTGCAAAGCAAGGAA | 122 |
| differen- | 001014382.2 | TGTGAAGGATGCTGG | |
| tiation | | GGAACTCTTT | |
| primary | | | |
| response | | | |
| gene | | | |
| (MYD88) | | | |
| TCDD- | NM_ | TAGTGCAGCCTGCTT | 176 |
| inducible | 001206048.1 | CTCCTAACCCCATCA | |
| poly | | AGTGAGCCAG | |
| (ADP- | | | |
| ribose) | | | |
| polymerase | | | |
| (TIRAP) | | | |
| TNF | NM_ | GGACTGCAGCAAAAGA | 156 |
| receptor- | 001034661.2 | CGACCTTCCCGCAAAG | |
| associated | | CCATCAAG | |
| factor 6, | | | |
| E3 ubi- | | | |
| quitin | | | |
| protein | | | |
| ligase | | | |
| (TRAF6) | | | |
| Inter- | NM_ | ACAGCATCAACATAC | 213 |
| leukin-1 | 001075998.1 | GTGCGGGTGCCCCAG | |
| receptor- | | TCAAACAGTA | |
| associated | | | |
| kinase 4 | | | |
| (IRAK4) | | | |
| Inter- | NM_ | GCCGCCCAGATCTAC | 233 |
| leukin-1 | 001040555.1 | AAGAATAGGAGTTCT | |
| receptor- | | CTTGCGGGGA | |
| associated | | | |
| kinase 1 | | | |
| (IRAK1) | | | |
| V-rel | NM_ | ACAGCTTTCAGAACC | 140 |
| reticu- | 001080242.2 | TGGGGGACGGCATTC | |
| loendo- | | AGGTCGTAG | |
| thellosis | | | |
| viral | | | |
| oncogene | | | |
| homolog | | | |
| A (avian) | | | |
| (NF-kappa- | | | |
| B p65 | | | |
| subunit) | | | |
| (RELA) | | | |

Complement system

| | | | |
|---|---|---|---|
| Complement | NM_ | AAGTTCATCACCCAC | 191 |
| component | 001040469 | ATCAAGCACTGTTTC | |
| 3 (C3) | | TGGTTCTCCTC | |

Chemokines

| | | | |
|---|---|---|---|
| Chemokine | NM_ | TCTCGCTGCAACATG | 121 |
| (C-C | 174006.2 | AAGGTTATAGCAGCA | |
| motif) | | GGCGACTTGG | |
| ligand 2 | | | |
| (CCL2) | | | |
| Chemokine | NM_ | TCCATGGCAGCAGTT | 129 |
| (C-C | 175827.2 | GTCTTTTCAGGTTCA | |
| motif) | | AGGCGTCCTC | |
| ligand 5 | | | |
| (CCL5) | | | |
| Chemokine | NM_ | CTTGTGGGCTTCACA | 115 |
| (C-C | 174263.2 | CAGCGTTTCACCCAC | |
| motif) | | TTCTTCTTTGG | |
| ligand 20 | | | |
| (CCL20) | | | |
| Chemokine | NM_ | TTGTGAGAGAGCTGC | 150 |
| (C-x-C | 174300.2 | GTTGTCCAGACAGAC | |
| motif) | | TTCCCTTCCA | |
| ligand 5 | | | |
| (CXCL5) | | | |
| Inter- | NM_ | AAGAATGAGTACAGA | 160 |
| leukin 8 | 173925.2 | ACTTCGATGCGTTTA | |
| (CXCL8) | | GGCAGACCTCGTTTC | |
| | | C | |
| Chemokine | NM_ | TCAACCCTGAAGCTC | 198 |
| (C-x-C | 001046513.2 | CCATGAGTCCAGCAC | |
| motif) | | ATCAAGTCCTT | |
| ligand 3 | | | |
| (CXCL3) | | | |
| Chemokine | NM_ | ATCATTGCTGTGGTC | 183 |
| (C-C | 001024930.3 | GTGGTGAAAGGGTTG | |
| motif) | | ACACAGCAGC | |
| receptor | | | |
| 7 (CCR7) | | | |
| Inter- | NM_ | CAGGTTGAGGCTGGA | 193 |
| leukin 13 | 001206677.1 | AGACACCCACCACTG | |
| receptor, | | CCATCTAAGT | |
| alpha 1 | | | |
| (IL13RA) | | | |

Inflammatory cytokines

| | | | |
|---|---|---|---|
| Inter- | NM_ | AGAATGTGGTGATGG | 224 |
| leukin 1, | 174092.1 | TGGCAACTTTGATTG | |
| alpha | | AGGGCGTCGT | |
| (IL1-A) | | | |
| Inter- | NM_ | GAAGAAAGGCCCGTC | 176 |
| leukin 1, | 174093.1 | TTCCTACAGTGAAGT | |
| beta | | TCAGGCTGCA | |
| (IL1-B) | | | |
| Inter- | NM_ | TGGTGATGACTTCTG | 109 |
| leukin 6 | 173923.2 | CTTTCCAGAGCTTCG | |
| (IL6) | | GTTTTCTCTGG | |
| Inter- | NM_ | AGCTGTATCCACTTG | 119 |
| leukin 10 | 174088.1 | CCAACCTGGGTCAAC | |
| (IL10) | | AGTAAGCTGTGC | |
| Tumor | NM_ | CCACGTTGTAGCCGA | 108 |
| necrosis | 173966.2 | CATCACCACCAGCTG | |
| factor α | | GTTGTCTTC | |
| (TNFα) | | | |
| Trans- | NM_ | CCTGGACACCAACTA | 185 |
| forming | 001166068.1 | CTGCTCCAGGACCTT | |
| growth | | GCTGTACTGT | |
| factor, | | | |
| beta 1 | | | |
| (TGFβ1) | | | |

Acute phase proteins/danger associated
molecular pattern molecules

| | | | |
|---|---|---|---|
| Serum | NM_ | CACGGGCATCATTTT | 179 |
| amyloid | 001242573.1 | CTGCTTGGGCACCGT | |
| A3 (SAA3) | | CATAGTTTCCA | |
| haptoglo- | NM_ | AATGAACGATGGGTC | 176 |
| bin (HP) | 001040470.1 | CTCACTTGATGAGCC | |
| | | CAATGTCTACC | |
| S100 | NM_ | CTGGTGCAAAAAGAG | 128 |
| calcium | 001046328.1 | CTGCAGCATAATGAA | |
| binding | | CTCCTCGAAGC | |
| protein | | | |
| A9 | | | |
| (S100A9) | | | |
| S100 | NM_ | TGGGGAGGCGCTGCT | 135 |
| calcium | 174651.2 | CTAGACTCGAAATGC | |
| binding | | CCCACCCGAACG | |
| protein | | | |
| A12 | | | |
| (S100A12) | | | |

Antimicrobial peptides

| | | | |
|---|---|---|---|
| Lactofer- | NM_ | CGAAGTGTGGATGGC | 215 |
| rin (LF) | 180998.2 | AAGGAATTCAAGGTG | |
| | | GTCAAGTAGCGG | |
| Lysozyme | NM_ | AAGAAACTTGGATTG | 185 |
| 1 K | 001077829.1 | GATGGCACTGCTTTT | |
| (LYZ1) | | GGGGTTTTGC | |
| Lactoper- | NM_ | TGGCTGTCAACCAAG | 134 |
| oxidase | 173933.2 | AAGCTGAGGCTCGAA | |
| (LPO) | | AATCTCCC | |
| Tracheal | NM_ | AGGAGTAGGAAATCC | 113 |
| antimi- | 174776.1 | TGTAAGCTGTGTAGC | |
| crobial | | ATTTTACTGCCCGCC | |
| peptide | | CGA | |
| (TAP) | | | |
| Lingual | NM_ | AGAAATTCTCAAAGC | 107 |
| antimi- | 203435.3 | TGCCGCAGCATTTTA | |
| crobial | | CTTGGGCTCC | |
| peptide | | | |
| (LAP) | | | |

Apoptosis

| | | | |
|---|---|---|---|
| Fas cell | NM_ | CGGGATCTGGGTTCA | 180 |
| surface | 174662.2 | CTTGTGGAGGACAAG | |
| death | | GCTGACAACA | |
| receptor | | | |
| (FAS) | | | |
| Tumor | NM_ | CGCCTCTGTCGTCTT | 170 |
| necrosis | 174674.2 | AGCATGACTGGAACT | |
| factor | | TGGGGTGGAG | |
| receptor | | | |
| super- | | | |
| family, | | | |
| member 1A | | | |
| (TNFRSF1A) | | | |
| Tumor | AF031589.1 | CCAGCAGCACGGACA | 153 |
| necrosis | | AGACAATGCAGGTGA | |
| factor | | CGTTGACC | |
| receptor 2 | | | |
| (TNER2) | | | |
| Caspase 8 | NM_ | TAGCATAGCACGGAA | 295 |
| (GASP8) | 001045970.2 | GCAGGGCCAGTGAAG | |
| | | TAAGAGGTCAG | |
| Caspase 3 | NM_ | TCAGTCAGTCAGTTG | 164 |
| (CASP3) | 001077840.1 | GGCACGGGAGCATCT | |
| | | TCCACACACA | |
| Caspase 1 | XM_ | ACGTCTTGCCCTTAT | 204 |
| (CASP1) | 002692921 | TATCTGCGTACTGTC | |
| | | AGAGGTCCGATGC | |
| BCL2-as- | NM_ | AGAGGATGATCGCAG | 200 |
| sociated | 173894.1 | CTGTGGAAGTCCAAT | |
| X | | GTCCAGCCCA | |
| protein | | | |
| (BAX) | | | |
| Anti- | AF245487 | GGCATTCAGCGACCT | 203 |
| apoptotic | | GACCCATCCAAGTTG | |
| regulator | | CGATCC | |
| Bcl-xL | | | |
| (Bcl-xL) | | | |
| B-cell | NM_ | ATGTGTGTGGAGAGC | 195 |
| CLL/ | 001166486.1 | GTCAAGAGCAGTGCC | |
| lymphoma | | TTCAGAGACA | |
| 2 (BCL-2) | | | |

Immunoglobulin receptors

| | | | |
|---|---|---|---|
| IgG Fc | AF141017.1 | GAGCTGGCTCCTTGG | 194 |
| receptor | | ATCTCATACCAGGAT | |
| (FcRN) | | TCCCGGAGGT | |
| Polymeric | NM_ | GACACCGTGGAGAGC | 192 |
| immuno- | 174143.1 | AAAGAGTGATTCGGA | |
| globulin | | GCGTGATTGC | |
| receptor | | | |
| (PIGR) | | | |

Scavenger Receptor

| | | | |
|---|---|---|---|
| CD68 | NM_ | GGCTCCAAGGAGGCA | 201 |
| molecule | 001045902.1 | ATAGGAATGAGAGGA | |
| (CD68) | | GCAAGTGGG | |
| CD40 | NM_ | TCGAAGGCCAACACT | 197 |
| molecule | 001105611.2 | GTACCGCCTTTTCTC | |
| (CD40) | | TCGCAGCTTG | |

JAK-STAT signaling

| | | | |
|---|---|---|---|
| Signal | NM_ | TCCTGCTGCGCTTTA | 213 |
| transducer | 001205689.1 | GTGAAGGATTCGCGG | |
| and acti- | | GTAGAGGAAG | |
| vator of | | | |
| transcrip- | | | |
| tion 2 | | | |
| (STAT2) | | | |

Oxidative metabolism

| | | | |
|---|---|---|---|
| Cytochrome | NM_ | GGACTTTGACCCAAC | 159 |
| P450, | 001192294.1 | CCGATCACTGGTGAG | |
| family 1, | | CAAGGATGGA | |
| subfamily | | | |
| B, | | | |
| polypep- | | | |
| tide 1 | | | |
| (CYP1B1) | | | |
| Cytochrome | AF514290.1 | GGAGCCTAAAACCCA | 177 |
| P450, | | CAGACACAGCACAAC | |
| family 1, | | TTTGGAAGGGC | |
| subfamily | | | |
| A, | | | |
| polypep- | | | |
| tide 1 | | | |
| (CYP1A1) | | | |
| Nitric | NM_ | CATTCGATGTCAGCG | 174 |
| oxide | 001076799.1 | GCAAGGCTGCGATTT | |
| synthase | | GAGCCTCATG | |
| 2, | | | |
| inducible | | | |
| (NOS2) | | | |

MAPK signaling

| | | | |
|---|---|---|---|
| FBJ murine | NM_ | ACTGCTCGCGATCAT | 173 |
| osteosar- | 182786.2 | GATGTCCAGATCGGT | |
| coma viral | | GCAGTAGTCC | |
| oncogene | | | |
| homolog | | | |
| (FOS) | | | |
| Mitogen- | NM_ | TGGAGGGGTAAAGGG | 156 |
| activated | 001192974.1 | CATTGAGAAACGGCC | |
| protein | | AGGAAGTGTT | |
| kinase 8 | | | |
| (MAPK8) | | | |

Others

| | | | |
|---|---|---|---|
| Matrix | NM_ | TCTGGAGCAATGTCA | 151 |
| metallopep- | 174112 | CACCCCCTGCACCTG | |
| tidase 1 | | GTTGAAAAGC | |
| (MMP1) | | | |
| Interferon | NM_ | GCTCAACTGACGGGA | 128 |
| regulatory | 001029845.2 | AGTGGTTTGGGTTCC | |
| factor | | CATGGTCTGG | |
| 3 (IRF3) | | | |
| Myxovirus | NM_ | AAGGCCACTATCCCC | 277 |
| (influenza | 173940.2 | TGCCTCGTACTTTGG | |
| virus) | | TAAACAGTCGG | |
| resistance | | | |
| 1, | | | |
| interferon- | | | |
| inducible | | | |
| protein | | | |
| p78 (mouse) | | | |
| (MX1) | | | |
| Myxovirus | NM_ | CTTCAGAGACGCCTC | 232 |
| (influenza | 173941.2 | AGTCGTGAAGGAGCC | |
| virus) | | AGGAATAGTG | |
| resistance | | | |
| 2 | | | |
| (mouse) | | | |
| (MX2) | | | |
| Nucleotide- | NM_ | CTGGCTCCGAGGAAA | 158 |
| binding | 001002889.1 | CACTTGTGCTCAGAT | |
| oligomer- | | GTCGTCCCAT | |
| ization | | | |
| domain | | | |
| containing | | | |
| 2 (NOD2) | | | |
| V-akt | NM_ | GATCACCGACTTCGG | 202 |
| murine | 173986.2 | ACTGTCTTCTCGTGG | |
| thymoma | | TCCTGGTTGT | |
| viral | | | |
| oncogene | | | |
| homolog 1 | | | |
| (AKT1) | | | |
| Wingless- | XM_ | CGGCCTTCACAGTGA | 150 |
| type MMTV | 010826681.1 | CTCTTGGCCTAGGAC | |
| integration | | AGTGTTTGCT | |
| site | | | |
| family | | | |
| member 4 | | | |
| (WNT4) | | | |

Reference genes

| | | | |
|---|---|---|---|
| 18S ribo- | AF176811.1 | CGGGGAGGTAGTGAC | 195 |
| somal RNA | | GAAACCGCTCCCAAG | |
| gene | | ATCCAACTA | |
| (18SrRNA) | | | |
| H3 histone, | NM_ | ACTTGCTACAAAAGC | 232 |
| family | 001014389.2 | CGCTCACTTGCCTCC | |
| 3A (H3F3A) | | TGCAAAGCAC | |
| Actin, | NM_ | AACTCCATCATGAAG | 234 |
| gamma 1 | 001033618 | TGTGACGATCCACAT | |
| (ACTG1) | | CTGCTGGAAGG | |
| Glyceral- | NM_ | GTCTTCACTACCATG | 197 |
| dehyd-3- | 001034034.1 | GAGAAGGTCATGGAT | |
| phosphate | | GACCTTGCCCAG | |
| dehydro- | | | |
| genase | | | |
| (GAPDH) | | | |
| Tyrosine | NM_ | CAGGCTGAGCGATAT | 141 |
| 3- | 174814.2 | GATGAGACCCTCCAA | |
| monoxy- | | GATGACCTAC | |
| genase/ | | | |
| tryptophan | | | |
| 5-monoxy- | | | |
| genase | | | |
| activa- | | | |
| tion | | | |
| protein, | | | |
| zeta | | | |
| poly- | | | |
| peptide | | | |
| (YWHAZ) | | | |
| Cytoker- | NM_ | TGGTGGAGGACTTCA | 215 |
| atin 8 | 001033610 | AGACCCGTGTCAGAA | |
| (KRT8) | | ATCTGAGACTGC | |
| Ubiquitine | NM_ | AGATCCAGGATAAGG | 426 |
| B (UBB) | 174133.2 | GAAGGCATCTCCACC | |
| | | TCCAGGGTGAT | |

¹L = Length

SUPPLEMENTARY TABLE 2
Fold changes in gene expression upon C. diff. treatment - statistical evaluation of the treatment
and time-effect with a paired t-test. High responder (n = 5), low responder (n = 4)

| | time point | | | | | |
|---|---|---|---|---|---|---|
| | C. diff. 6 h¹ | | C. diff. 24 h¹ | | C. diff. 72 h¹ | |
| genes | High² | Low³ | High² | Low³ | High² | Low³ |

TLR pathway

| | | | | | | | | | | | | | |
|---|---|---|---|---|---|---|---|---|---|---|---|---|---|
| TLR2 | Fold | 1.51 | | 1.14 | | 1.29 | | 1.34 | | 1.40 | | 1.08 | |
| | SEM | 0.37 | | 0.35 | | 0.21 | | 0.24 | | 0.22 | | 0.19 | |
| TLR4 | Fold | 1.24 | | 1.10 | | 1.03 | | 0.87 | + | 0.87 | * | 1.02 | |
| | SEM | 0.16 | | 0.06 | | 0.09 | | 0.08 | | 0.08 | | 0.13 | |
| LY96 | Fold | 1.05 | A | 0.91 | +a | 1.19 | +B | 1.03 | b | 0.89 | +B | 1.01 | b |
| | SEM | 0.11 | | 0.05 | | 0.08 | | 0.09 | | 0.07 | | 0.13 | |
| LBP | Fold | 1.65 | | 1.50 | a | 0.96 | + | 1.57 | ab | 0.78 | * | 0.81 | +b |
| | SEM | 0.60 | | 0.46 | | 0.25 | | 0.59 | | 0.20 | | 0.12 | |
| CD14 | Fold | 1.06 | A | 0.99 | a | 1.70 | +B | 0.78 | ab | 1.64 | B | 1.05 | b |
| | SEM | 0.29 | | 0.10 | | 0.34 | | 0.14 | | 0.36 | | 0.10 | |
| MYD88 | Fold | 1.03 | A | 1.28 | *a | 1.18 | +A | 1.04 | b | 0.95 | B | 0.91 | *c |
| | SEM | 0.09 | | 0.09 | | 0.08 | | 0.05 | | 0.09 | | 0.08 | |
| TIRAP | Fold | 1.53 | *** | 1.19 | *a | 1.25 | ** | 1.04 | a | 1.15 | | 0.99 | b |
| | SEM | 0.12 | | 0.07 | | 0.07 | | 0.08 | | 0.09 | | 0.08 | |
| TRAF6 | Fold | 1.30 | **A | 1.22 | ***a | 1.14 | AB | 0.91 | ab | 1.00 | B | 0.91 | b |
| | SEM | 0.07 | | 0.05 | | 0.11 | | 0.07 | | 0.09 | | 0.08 | |
| IRAK4 | Fold | 1.11 | A | 1.00 | a | 1.12 | B | 1.13 | ab | 1.38 | A | 1.17 | b |
| | SEM | 0.16 | | 0.11 | | 0.10 | | 0.21 | | 0.20 | | 0.17 | |
| IRAK1 | Fold | 1.06 | A | 1.00 | | 1.14 | *B | 1.13 | | 0.95 | B | 1.17 | |
| | SEM | 0.08 | | 0.11 | | 0.05 | | 0.21 | | 0.07 | | 0.17 | |
| RELA | Fold | 1.22 | *A | 1.27 | *a | 1.11 | A | 0.97 | b | 1.23 | B | 0.80 | +c |
| | SEM | 0.08 | | 0.10 | | 0.06 | | 0.06 | | 0.32 | | 0.12 | |

Complement system

| | | | | | | | | | | | | | |
|---|---|---|---|---|---|---|---|---|---|---|---|---|---|
| C3 | Fold | 2.13 | A | 1.36 | a | 0.42 | ***A | 0.88 | a | 0.65 | * * B | 0.90 | b |
| | SEM | 0.70 | | 0.51 | | 0.09 | | 0.26 | | 0.15 | | 0.19 | |

Chemokines

| | | | | | | | | | | | | | |
|---|---|---|---|---|---|---|---|---|---|---|---|---|---|
| CCL5 | Fold | 1.47 | | 0.72 | **a | 1.16 | | 0.94 | a | 0.74 | * | 0.99 | b |
| | SEM | 0.27 | | 0.11 | | 0.16 | | 0.17 | | 0.21 | | 0.12 | |
| CCL20 | Fold | 1.14 | A | 1.30 | a | 0.53 | ***A | 0.52 | **a | 2.08 | **B | 0.94 | b |
| | SEM | 0.21 | | 0.21 | | 0.06 | | 0.10 | | 0.32 | | 0.12 | |
| CXCL5 | Fold | 1.30 | A | 1.10 | a | 0.97 | *B | 1.12 | b | 1.21 | A | 1.14 | c |
| | SEM | 0.20 | | 0.10 | | 0.12 | | 0.16 | | 0.18 | | 0.32 | |
| CXCL8 | Fold | 2.01 | ***A | 1.31 | | 0.84 | *A | 0.84 | + | 1.41 | B | 0.98 | |
| | SEM | 0.22 | | 0.16 | | 0.10 | | 0.12 | | 0.27 | | 0.13 | |
| CXCL3 | Fold | 1.56 | *A | 1.5 | **a | 1.14 | B | 1.17 | b | 1.23 | B | 1.19 | b |
| | SEM | 0.16 | | 0.16 | | 0.10 | | 0.14 | | 0.17 | | 0.30 | |
| CCR7 | Fold | 1.43 | A | 0.88 | +a | 2.35 | B | 2.85 | **b | 0.66 | **A | 0.95 | ab |
| | SEM | 0.37 | | 0.23 | | 0.74 | | 0.74 | | 0.14 | | 0.15 | |
| IL13RA | Fold | 1.12 | A | 1.09 | a | 1.20 | +B | 1.02 | ab | 1.12 | B | 0.89 | b |
| | SEM | 0.07 | | 0.08 | | 0.09 | | 0.12 | | 0.08 | | 0.08 | |

Inflammatory cytokines

| | | | | | | | | | | | | | |
|---|---|---|---|---|---|---|---|---|---|---|---|---|---|
| IL1-A | Fold | 1.18 | | 0.97 | a | 0.76 | ** | 0.71 | **ab | 2.51 | *** | 1.04 | b |
| | SEM | 0.17 | | 0.08 | | 0.06 | | 0.07 | | 0.54 | | 0.12 | |
| IL6 | Fold | 0.43 | **A | 0.70 | **a | 1.52 | ***B | 1.27 | *b | 2.00 | A | 1.01 | a |
| | SEM | 0.07 | | 0.08 | | 0.12 | | 0.10 | | 0.63 | | 0.13 | |
| TNFα | Fold | 0.91 | | 1.31 | | 0.86 | | 0.80 | * | 2.25 | | 1.14 | |
| | SEM | 0.16 | | 0.22 | | 0.14 | | 0.12 | | 0.57 | | 0.24 | |
| TGFβ1 | Fold | 0.94 | | 1.18 | * | 0.85 | * | 0.93 | | 0.76 | ** | 0.97 | |
| | SEM | 0.06 | | 0.07 | | 0.08 | | 0.08 | | 0.09 | | 0.10 | |

Acute phase proteins/danger associated molecular pattern molecules

| | | | | | | | | | | | | | |
|---|---|---|---|---|---|---|---|---|---|---|---|---|---|
| SAA3 | Fold | 2.77 | AB | 1.26 | a | 0.57 | ***A | 1.52 | ab | 1.08 | B | 1.25 | b |
| | SEM | 1.06 | | 0.26 | | 0.22 | | 0.68 | | 0.28 | | 0.43 | |
| S100A9 | Fold | 1.16 | A | 1.25 | a | 1.70 | ***B | 1.52 | **b | 2.26 | +C | 0.99 | c |
| | SEM | 0.11 | | 0.16 | | 0.19 | | 0.16 | | 0.56 | | 0.16 | |
| S100A12 | Fold | 1.26 | A | 0.87 | + | 2.20 | ***B | 1.71 | + | 2.02 | *B | 0.95 | |
| | SEM | 0.22 | | 0.07 | | 0.29 | | 0.24 | | 0.56 | | 0.19 | |

Antimicrobial peptides

| | | | | | | | | | | | | | |
|---|---|---|---|---|---|---|---|---|---|---|---|---|---|
| LF | Fold | 1.53 | A | 1.16 | a | 0.62 | ***B | 1.21 | b | 0.69 | *B | 0.78 | *B |
| | SEM | 0.42 | | 0.12 | | 0.09 | | 0.37 | | 0.15 | | 0.13 | |
| LYZ1 | Fold | 1.90 | A | 0.97 | a | 0.71 | **B | 1.10 | a | 3.04 | ***C | 1.65 | *b |
| | SEM | 0.39 | | 0.10 | | 0.09 | | 0.17 | | 0.72 | | 0.36 | |
| LPO | Fold | 1.16 | | 1.06 | | 1.38 | | 1.04 | | 1.58 | * | 1.02 | |
| | SEM | 0.15 | | 0.10 | | 0.24 | | 0.19 | | 0.23 | | 0.15 | |

Apoptosis

| | | | | | | | | | | | | | |
|---|---|---|---|---|---|---|---|---|---|---|---|---|---|
| FAS | Fold | 1.21 | *A | 1.21 | **a | 1.82 | *B | 0.93 | a | 1.42 | *A | 0.89 | +b |
| | SEM | 0.08 | | 0.07 | | 0.40 | | 0.09 | | 0.16 | | 0.07 | |
| TNFRSF1A | Fold | 1.10 | | 1.35 | *a | 1.08 | | 0.91 | ab | 1.01 | | 0.97 | b |
| | SEM | 0.09 | | 0.15 | | 0.09 | | 0.09 | | 0.12 | | 0.12 | |
| TNFR2 | Fold | 1.17 | | 1.10 | | 0.91 | | 0.92 | | 0.84 | * | 1.06 | |
| | SEM | 0.12 | | 0.14 | | 0.09 | | 0.12 | | 0.13 | | 0.19 | |
| CASP8 | Fold | 1.14 | A | 1.09 | | 1.24 | B | 0.93 | b | 1.07 | C | 1.02 | b |
| | SEM | 0.11 | | 0.05 | | 0.12 | | 0.08 | | 0.12 | | 0.11 | |
| CASP3 | Fold | 1.27 | A | 1.08 | a | 0.99 | B | 0.89 | b | 0.91 | +-B | 0.96 | b |
| | SEM | 0.13 | | 0.10 | | 0.07 | | 0.09 | | 0.14 | | 0.11 | |
| BAX | Fold | 1.15 | +A | 1.00 | a | 1.05 | A | 1.00 | a | 1.56 | B | 1.33 | b |
| | SEM | 0.10 | | 0.04 | | 0.05 | | 0.05 | | 0.30 | | 0.25 | |
| BCL-xL | Fold | 0.87 | | 1.01 | | 1.04 | | 0.92 | | 1.18 | | 1.07 | |
| | SEM | 0.07 | | 0.07 | | 0.07 | | 0.08 | | 0.10 | | 0.13 | |
| BCL-2 | Fold | 0.58 | ***A | 1.17 | a | 0.77 | *AB | 0.79 | *b | 0.83 | | 1.11 | b |
| | SEM | 0.06 | | 0.18 | | 0.11 | | 0.07 | | 0.12 | | 0.17 | |

Immunoglobulin receptors

| | | | | | | | | | | | | | |
|---|---|---|---|---|---|---|---|---|---|---|---|---|---|
| FcRN | Fold | 1.37 | | 0.96 | a | 1.09 | | 1.05 | b | 0.81 | * | 1.04 | ab |
| | SEM | 0.39 | | 0.19 | | 0.13 | | 0.12 | | 0.09 | | 0.17 | |
| PIGR | Fold | 0.89 | A | 0.90 | | 0.83 | +B | 0.70 | ** | 0.56 | **AB | 0.62 | * |
| | SEM | 0.15 | | 0.16 | | 0.12 | | 0.09 | | 0.15 | | 0.14 | |

Scavenger Receptor

| | | | | | | | | | | | | | |
|---|---|---|---|---|---|---|---|---|---|---|---|---|---|
| CD68 | Fold⁴ | 1.84 | **A | 1.38 | a | 1.38 | B | 0.92 | b | 3.87 | +C | 1.24 | c |
| | SEM | 0.29 | | 0.30 | | 0.19 | | 0.12 | | 2.31 | | 0.34 | |
| CD40 | Fold | 1.12 | | 1.08 | a | 0.97 | | 0.88 | b | 1.27 | | 1.05 | ab |
| | SEM | 0.08 | | 0.05 | | 0.04 | | 0.10 | | 0.14 | | 0.14 | |

JAK-STAT signaling

| | | | | | | | | |
|---|---|---|---|---|---|---|---|---|
| STAT2 | Fold | 1.20 | 1.15 | * | 1.21 | 0.94 | 1.06 | 1.08 |
| | SEM | 0.16 | 0.06 | | 0.11 | 0.11 | 0.12 | 0.13 |

Oxidative metabolism

| | | | | | | | | | | | | | |
|---|---|---|---|---|---|---|---|---|---|---|---|---|---|
| CYP1B1 | Fold | 1.08 | A | 1.15 | a | 1.06 | B | 1.17 | b | 2.09 | c | 1.25 | c |
| | SEM | 0.11 | | 0.08 | | 0.09 | | 0.12 | | 0.51 | | 0.21 | |
| NOS2 | Fold | 1.55 | A | 1.19 | a | 1.09 | AB | 1.66 | a | 1.33 | B | 1.28 | b |
| | SEM | 0.30 | | 0.20 | | 0.12 | | 0.38 | | 0.17 | | 0.21 | |

MAPK signaling

| | | | | | | | | | | | | | |
|---|---|---|---|---|---|---|---|---|---|---|---|---|---|
| Fos | Fold | 1.28 | ***A | 1.25 | * | 0.91 | *B | 0.99 | | 1.01 | A | 1.00 | |
| | SEM | 0.06 | | 0.07 | | 0.05 | | 0.09 | | 0.12 | | 0.08 | |
| MAPK8 | Fold | 1.34 | A | 0.98 | a | 1.32 | + B | 1.01 | b | 0.74 | *C | 0.92 | a |
| | SEM | 0.23 | | 0.13 | | 0.14 | | 0.10 | | 0.11 | | 0.11 | |

Others

| | | | | | | | | | | | | | |
|---|---|---|---|---|---|---|---|---|---|---|---|---|---|
| MMP1 | Fold | 1.56 | ***A | 1.19 | **a | 2.22 | ***B | 1.06 | ab | 2.42 | **B | 0.95 | b |
| | SEM | 0.11 | | 0.06 | | 0.42 | | 0.10 | | 0.50 | | 0.07 | |
| IRF3 | Fold | 1.31 | A | 1.08 | a | 1.03 | B | 0.95 | a | 1.02 | B | 1.18 | b |
| | SEM | 0.31 | | 0.13 | | 0.10 | | 0.09 | | 0.18 | | 0.22 | |
| MX1 | Fold | 1.13 | A | 1.06 | a | 0.73 | B | 1.30 | b | 1.06 | C | 0.90 | b |
| | SEM | 0.18 | | 0.22 | | 0.11 | | 0.61 | | 0.16 | | 0.19 | |
| MX2 | Fold | 0.99 | A | 1.32 | a | 0.83 | +B | 1.27 | b | 1.19 | C | 0.54 | **b |
| | SEM | 0.20 | | 0.42 | | 0.14 | | 0.50 | | 0.16 | | 0.10 | |
| NOD2 | Fold | 0.94 | +A | 0.97 | a | 1.04 | B | 0.94 | b | 1.15 | C | 0.72 | *c |
| | SEM | 0.24 | | 0.11 | | 0.28 | | 0.11 | | 0.21 | | 0.14 | |
| AKT1 | Fold | 1.09 | | 1.12 | a | 0.83 | ** | 0.91 | +a | 0.97 | | 0.96 | b |
| | SEM | 0.08 | | 0.11 | | 0.06 | | 0.06 | | 0.16 | | 0.13 | |
| WNT4 | Fold | 0.92 | A | 1.78 | | 1.10 | B | 1.22 | | 0.98 | B | 0.63 | ** |
| | SEM | 0.17 | | 0.55 | | 0.17 | | 0.25 | | 0.19 | | 0.10 | |

¹Treatment time with C. diff. in hours
²High = High responder animals (n = 5)
³Low = Low responder animals (n = 4)
⁴Fold = Fold Change [2\hat{ }^{(-ΔΔCq)}]
* p ≤ 0.05,
** p ≤ 0.01,
*** p ≤ 0.001 between treatment and control
+ Distinct changes (0.01 ≤ p < 0.05) between treatment and control
Upper case letter: significant changes between treatment time-points high responder animals
Lower case letter: significant changes between treatment time-points low responder animals

### REFERENCES

1. Poutanen S M. Clostridium difficile-associated diarrhea in adults. CMAJ. 2004; 171: 51-58. doi: 10.1503/cmaj.1031189.
2. Onal C M, Steinert M. Novel therapeutic strategies for Clostridium difficile infections. Expert Opin Ther Targets. 2016; 20: 269-285. doi: 10.1517/14728222.2016.1090428.
3. Viswanathan V K, Mallozzi M J, Vedantam G. Clostridium difficile infection: An overview of the disease and its pathogenesis, epidemiology and interventions. Gut Microbes. 2010; 1: 234-242. doi: 10.4161/gmic.1.4.12706.
4. Larrainzar-Coghen T, Rodriguez-Pardo D, Puig-Asensio M, Rodriguez V, Ferrer C, Bartolomb R, et al. First recurrence of Clostridium difficile infection: clinical relevance, risk factors, and prognosis. Eur J Clin Microbiol Infect Dis. 2016; 35: 371-378. doi: 10.1007/s10096-015-2549-9.
5. Elliott B, Chang B J, Golledge C L, Riley T V. Clostridium difficile-associated diarrhoea. Intern Med J. 2007; 37: 561-568. doi: 10.1111/j.1445-5994.2007.01403.x.
6. Bagdasarian N, Rao K, Malani P N. Diagnosis and treatment of Clostridium difficile in adults: a systematic review. JAMA. 2015; 313: 398-408. doi: 10.1001/jama.2014.17103.
7. Mullane K. Fidaxomicin in Clostridium difficile infection: latest evidence and clinical guidance. Ther Adv Chronic Dis. 2014; 5: 69-84. doi: 10.1177/2040622313511285.
8. Louie T J, Miller M A, Mullane K M, Weiss K, Lentnek A, Golan Y, et al. Fidaxomicin versus vancomycin for Clostridium difficile infection. N Engl J Med. 2011; 364: 422-431. doi: 10.1056/NEJMoa0910812.
9. Van Dissel J T, De Groot N, Hensgens C M, Numan S, Kuijper E J, Veldkamp P, et al. Bovine antibody-enriched whey to aid in the prevention of a relapse of Clostridium difficile-associated diarrhoea: preclinical and preliminary clinical data. J Med Microbiol. 2005; 54: 197-205. doi: 10.1099/jmm.0.45773-0.
10. Hermann C. Review: variability of host-pathogen interaction. J Endotoxin Res. 2007; 13: 199-218. doi: 10.1177/0968051907082605.
11. Gunther J, Koczan D, Yang W, Nurnberg G, Repsilber D, Schuberth H J, Park Z, Maqbool N, Molenaar A, Seyfert H M. Assessment of the immune capacity of mammary epithelial cells: comparison with mammary tissue after challenge with Escherichia coli. Vet Res 2009; 40: 31. doi: 10.1051/vetres/2009014.
12. Sorg D, Danowski K, Korenkova V, Rusnakova V, Kiffner R, Zimmer R, Meyer H H D, Kliem H. Microfluidic high-throughput RT-qPCR measurements of the immune response of primary bovine mammary epithelial cells cultured from milk to mastitis pathogens. Animal. 2013; 7: 799-805. doi: 10.1017/S1751731112002315.
13. Strandberg Y, Gray C, Vuocolo T, Donaldson L, Broadway M, Tellam R. Lipopolysaccharide and lipoteichoic acid induce different innate immune responses in bovine mammary epithelial cells. Cytokine. 2005; 31: 72-86. doi: 10.1016/j.cyto.2005.02.010.
14. Griesbeck-Zilch B, Meyer H, Kühn C, Schwerin M, Wellnitz O. Staphylococcus aureus and Escherichia coli cause deviating expression profiles of cytokines and lactoferrin messenger ribonucleic acid in mammary epithelial cells. J Dairy Sci. 2008; 91: 2215-2224. doi: 10.3168/jds.2007-0752.
15. Prakash B S, Meyer H H, Schallenberger E, Van de Wiel D F. Development of a sensitive enzymeimmunoassay (EIA) for progesterone determination in unextracted bovine plasma using the second antibody technique. J Steroid Biochem. 1987; 28: 623-627.
16. Sorg D, Fandrey E, Frblich K, Meyer H, Kliem H. Mammary immunity of White Park and Highland cattle compared with Brown Swiss and Red Holstein. Anim Genet Resour. 2013; 52: 91-104. doi: 10.1017/S2078633612000781.
17. Danowski K, Gross J J, Meyer H H D, Kliem H. Effects of induced energy deficiency on lactoferrin concentration in milk and the lactoferrin reaction of primary bovine mammary epithelial cells in vitro. J Anim Physiol Anim Nutr (Berl). 2013; 97: 647-655. doi: 10.1111/j.1439-0396.2012.01305.x.
18. Wellnitz O, Arnold E T, Bruckmaier R M. Lipopolysaccharide and lipoteichoic acid induce different immune responses in the bovine mammary gland. J Dairy Sci. 2011; 94: 5405-5412. doi: 10.3168/jds.2010-3931.
19. Gilbert F B, Cunha P, Jensen K, Glass E J, Foucras G, Robert-Granie C, et al. Differential response of bovine mammary epithelial cells to Staphylococcus aureus or Escherichia coli agonists of the innate immune system. Vet Res. 2013; 44: 40. doi: 10.1186/1297-9716-44-40.
20. Koressaar T, Remm M. Enhancements and modifications of primer design program Primer3. Bioinformatics. 2007; 23: 1289-1291. doi: 10.1093/bioinformatics/btm091.
21. Untergasser A, Cutcutache I, Koressaar T, Ye J, Faircloth B C, Remm M, et al. Primer3—new capabilities and interfaces. Nucleic Acids Res. 2012; 40: e115. doi: 10.1093/nar/gks596.
22. Bustin S A, Benes V, Garson J A, Hellemans J, Huggett J, Kubista M, et al. The MIQE guidelines: Minimum information for publication of quantitative Real-Time PCR experiments. Clinical Chemistry. 2009; 55: 611-622. doi: 10.1373/clinchem.2008.112797
23. Livak K J, Schmittgen T D. Analysis of relative gene expression data using Real-Time Quantitative PCR and the 2-ΔΔCT method. Methods. 2001; 25: 402-408. doi: 10.1006/meth.2001.1262.
24. Saeed A I, Sharov V, White J, Li J, Liang W, Bhagabati N, et al. TM4: a free, open-source system for microarray data management and analysis. BioTechniques. 2003; 34: 374-378.
25. Dziarski R, Wang Q, Miyake K, Kirschning C J, Gupta D. MD-2 enables Toll-like receptor 2 (TLR2)-mediated responses to lipopolysaccharide and enhances TLR2-mediated responses to gram-positive and gram-negative bacteria and their cell wall components. J Immunol. 2001; 166: 1938-1944.
26. Lutzow Y, Strandberg C, Donaldson L, Gray C P, Vuocolo T, Pearson R D, Reverter A, et al. Identification of immune genes and proteins involved in the response of bovine mammary tissue to Staphylococcus aureus infection. BMC Vet Res. 2008; 4: 18. doi: 10.1186/1746-6148-4-18.
27. Aderem A, Ulevitch R J. Toll-like receptors in the induction of the innate immune response. Nature. 2000; 406: 782-787. doi: 10.1038/35021228.
28. Janeway C, Murphy K P. Janeway Immunologie. 7th ed. Heidelberg: Spektrum, Akad. Verl; 2009.
29. Boudjellab N, Chan-Tang H S, Zhao X. Bovine interleukin-1 expression by cultured mammary epithelial cells (MAC-T) and its involvement in the release of MAC-T derived interleukin-8. Comp Biochem Physiol, Part A Mol Integr Physiol. 2000; 127: 191-199.
30. Lahouassa H, Moussay E, Rainard P, Riollet C. Differential cytokine and chemokine responses of bovine mammary epithelial cells to Staphylococcus aureus and Escherichia coli. Cytokine. 2007; 38: 12-21. doi: 713 10.1016/j.cyto.2007.04.006.
31. Rainard P, Riollet C. Innate immunity of the bovine mammary gland. Vet. Res. 2006; 37: 369-400. doi: 10.1051/vetres:2006007.
32. Ryckman C, Vandal K, Rouleau P, Talbot M, Tessier P A. Proinflammatory Activities of S100: Proteins S100A8, S100A9, and S100A8/A9 Induce Neutrophil Chemotaxis and Adhesion. J Immunol. 2003; 170: 3233-3242. doi: 10.4049/jimmunol.170.6.3233.
33. Yang Z, Tao T, Raftery M J, Youssef P, Di Girolamo N, Geczy C L. Proinflammatory properties of the human S100 protein S100A12. J Leukoc Biol. 2001; 69: 986-994.
34. Wesson C A, Deringer J, Liou L E, Bayles K W, Bohach G A, Trumble W R. Apoptosis induced by Staphylococcus aureus in epithelial cells utilizes a mechanism involving caspases 8 and 3. Infect Immun. 2000; 68: 2998-3001.
35. Schmittgen T D, Livak K J. Analyzing real-time PCR data by the comparative C(T) method. Nat Protoc. 2008; 3(6):1101-8.
36. Pfaffl M W. A new mathematical model for relative quantification in real-time R T-PCR. Nucleic Acids Res. 2001 May 1; 29(9):e45.
37. Pfaffl M W, Horgan G W, Dempfle L. Relative expression software tool (REST) for group-wise comparison and statistical analysis of relative expression results in real-time PCR. Nucleic Acids Res. 2002 May 1; 30(9):e36.

1. A method of selecting or identifying a cow for C. difficile immunisation, the method comprising:
(a) determining expression level of a plurality of genes of interest in a cell obtained from a cow;
(b) stimulating the cell with C. difficile and/or a C. difficile specific antigen;
(c) determining the expression of the plurality of genes of interest in the stimulated cell of step (b); and
(d) selecting the cow where there is an at least a two-fold increase or decrease in expression of each of the plurality of genes of interest as determined in step (c) relative to step (a),
wherein the plurality of genes of interest are selected from the group consisting of: LF, TGFB1, CASP3, TIRAP, CXCL5, TLR2, SAA3, NOS2, CXCL8, AKT1, IRF3, C3, TNFR2, FOS, CXCL3, CCL5, CCR7, LY96, TRAF6, MYD88, STAT2, TLR4, TNFRSF1A, IL13RA, CASP8, MAPK8, IRAK1, S100A12, BCL-X, CCL20, NOD2, LPO, RELA, TNFα, LYZ1, BAX, IL6, CD40, WNT4, IL1-A, CD68, MMP1, IRAK4, FAS, CD14, S100A9, BCL2, CYP1B1, LBP, MX1 and MX2.

2. The method according to claim 1, wherein the plurality of genes of interest comprise at least: LY96, CD14 and TIRAP.

3. The method according to claim 1, wherein the plurality of genes of interest includes at least one gene from the “early set” consisting of: SAA3, LF, C3, TIRAP, CXCL5, CXCL3, CXCL8, TRAF6, RELA, CD14, CCL5, IL6, FAS, CASP3, BCL-2, CD68, CD40, MAPK8, TGFB1, STAT2, TLR4, AKT1, TNFRSF1A, WNT4, IRF3, TNFR2, TLR2, NOS2, LBP and FOS, and wherein the gene expression determination in step (c) for each gene from said early set is after stimulation of the cell in step (b) for between 2 and 22 hours.

4. The method according to claim 1, wherein the plurality of genes of interest includes at least one gene from the “intermediate set” consisting of: CCL5, CCR7, LY96, TRAF6, MYD88, STAT2, TLR4, TLR2, CD14, TIRAP, THFRSF1A, IL13RA, CASP8, CASP3, MAPK8, IRAK1, IRAK4, RELA, IL6, FAS, BAX, CD68, CD40, MMP1, AKT1, TGFB1 NOS2, LBP, MX1 and MX2, and wherein the gene expression determination in step (c) for each gene from said intermediate set is after stimulation of the cell in step (b) for between 22 and 48 hours.

5. The method according to claim 1, wherein the plurality of genes of interest includes at least one gene from the “late set” consisting of: IL6, IL1-A, IL13RA, S100A12, S100A9, LYZ1, LPO, CD14, FAS, CASP8, BAX, CD68, CD40, MMP1, NOD2, BCL-xL, CCL20, TNFα, IRAK4, CYP1B1, LY96, TIRAP, CXCL8, IL13RA, BCL2, RELA, MX2 and WNT4, and wherein the gene expression determination in step (c) for each gene from said intermediate set is after stimulation of the cell in step (b) for between 48 and 96 hours.

6. The method according to claim 1, wherein the plurality of genes of interest further comprises FcRn and/or pIGR.

7. A method of selecting or identifying a cow for C. difficile immunisation, the method comprising
(a) determining expression level of a plurality of genes of interest in a cell obtained from a cow;
(b) stimulating the cell with C. difficile and/or a C. difficile specific antigen;
(c) determining the expression of the plurality of genes of interest in the stimulated cell of step (b);
(d) comparing the change in expression of each of the plurality of genes of interest between step (a) and (c) with the expression change for each of the same genes of interest in a cow already determined to be a high responder and a cow already determined to be low responder cow, and
(e) selecting a cow if the change in expression levels of the genes determined in step (d) is more similar to the change for a high responder cow than a low responder cow, wherein the plurality of genes of interest are selected from the group consisting of: LF, TGFB1, CASP3, TIRAP, CXCL5, TLR2, SAA3, NOS2, CXCL8, AKT1, IRF3, C3, TNFR2, FOS, CXCL3, CCL5, CCR7, LY96, TRAF6, MYD88, STAT2, TLR4, TNFRSF1A, IL13RA, CASP8, MAPK8, IRAK1, S100A12, BCL-X, CCL20, NOD2, LPO, RELA, TNFα, LYZ1, BAX, IL6, CD40, WNT4, IL1-A, CD68, MMP1, IRAK4, FAS, CD14, S100A9, BCL2, CYP1B1, LBP, MX1 and MX2.

8. The method according to claim 7, wherein determining the expression level of each of said plurality of genes of interest comprises performing real-time quantitative reverse transcriptase polymerase chain reaction (PCR).

9. The method according to claim 8, wherein gene expression determination is a relative determination that derives a fold-change in expression of each of said plurality of genes of step (c) relative to step (a).

10. The method according to claim 9, wherein gene expression determination employs the 2^{-ΔΔCT} method.

11. The method according to claim 7, wherein the expression of each of said plurality of genes is normalised against expression levels of a panel of reference genes, said reference genes being other than innate immune system genes.

12. The method according to claim 11, wherein said reference genes are selected from the group consisting of: GAPDH, YWHAZ, H3F3A, ACTy1, 18srRNA, Cyt8 and UBB.

13. The method according to claim 1, wherein the method is an in vitro method carried out on a cell isolated from the cow, optionally wherein the cell is a cultured cell.

14. The method according to claim 1, wherein the cell is a primary bovine mammary epithelial cell or a lymphocyte.

15. The method according to claim 1, wherein the antigen comprises Toxin A and/or Toxin B.

16. The method according to claim 1, wherein the cells are stimulated with inactivated C. difficile.

17. The method according to claim 1, wherein the cow is selected for C. difficile immunisation based on the stimulation-induced change in expression of said plurality of genes, said method further comprising the step of immunising the selected cow with C. difficile vaccine.

18. The method according to claim 17, further comprising collecting milk from the immunised cow.

19. The method according to claim 18, wherein the collected milk, or C. difficile-specific antibodies concentrated from the collected milk, is administered to a patient having, or at risk of developing, a C. difficile infection or a C. difficile infection-related complication.

20. A method of immunising a cow, comprising administering a C. difficile vaccine to a cow that has been selected for C. difficile immunisation by a method as defined in claim 1, and has thereby been identified as being predisposed to be a high responder.

21. A method according to claim 20, further comprising collecting milk from the immunised cow.

22. A kit for use in a method as defined in claim 1, comprising a primer pair for each of at least 5 genes selection from the groups consisting of: LF, TGFB1, CASP3, TIRAP, CXCL5, TLR2, SAA3, NOS2, CXCL8, AKT1, IRF3, C3, TNFR2, FOS, CXCL3, CCL5, CCR7, LY96, TRAF6, MYD88, STAT2, TLR4, TNFRSF1A, IL13RA, CASP8, MAPK8, IRAK1, S100A12, BCL-X, CCL20, NOD2, LPO, RELA, TNFα, LYZ1, BAX, IL6, CD40, WNT4, IL1-A, CD68, MMP1, IRAK4, FAS, CD14, S100A9, BCL2, CYP1B1, LBP, MX1 and MX2.

23. The kit according to claim 22, wherein said primer pairs are selected from the primers set forth in Supplementary Table 1.

24. The method according to claim 1, wherein determining the expression level of each of said plurality of genes of interest comprises performing real-time quantitative reverse transcriptase polymerase chain reaction (PCR).

25. The method according to claim 1, wherein the expression of each of said plurality of genes is normalised against expression levels of a panel of reference genes, said reference genes being other than innate immune system genes.

26. A method of immunising a cow, comprising administering a C. difficile vaccine to a cow that has been selected for C. difficile immunisation by a method as defined in claim 7, and has thereby been identified as being predisposed to be a high responder.


SEQUENCE LISTING (136 sequences)

SEQ ID NO: 1 - DNA, 22 bp, Artificial Sequence
  Toll-like receptor 2 (TLR2)
cattcctggcaagtggattatc

SEQ ID NO: 2 - DNA, 22 bp, Artificial Sequence
  Toll-like receptor 2 (TLR2)
ggaatggccttcttgtcaatgg

SEQ ID NO: 3 - DNA, 20 bp, Artificial Sequence
  Toll-like receptor 4 (TLR4)
tgctggctgcaaaaagtatg

SEQ ID NO: 4 - DNA, 20 bp, Artificial Sequence
  Toll-like receptor 4 (TLR4)
ttacggcttttgtggaaacc

SEQ ID NO: 5 - DNA, 22 bp, Artificial Sequence
  Lymphocyte antigen 96 (LY96)
tgtttcaatacgttctgagccc

SEQ ID NO: 6 - DNA, 19 bp, Artificial Sequence
  Lymphocyte antigen 96 (LY96)
tcagtgttcccctcgatgg

SEQ ID NO: 7 - DNA, 20 bp, Artificial Sequence
  Lipopolysaccharide binding protein (LBP)
tcccagttgctttccttgct

SEQ ID NO: 8 - DNA, 20 bp, Artificial Sequence
  Lipopolysaccharide binding protein (LBP)
gcggaaggacttggtgttct

SEQ ID NO: 9 - DNA, 20 bp, Artificial Sequence
  CD14 molecule (CD14)
gcagcctggaacagtttctc

SEQ ID NO: 10 - DNA, 20 bp, Artificial Sequence
  CD14 molecule (CD14)
accagaagctgagcaggaac

SEQ ID NO: 11 - DNA, 20 bp, Artificial Sequence
  Myeloid differentiation primary response gene (MYD88)
ctgcaaagcaaggaatgtga

SEQ ID NO: 12 - DNA, 20 bp, Artificial Sequence
  Myeloid differentiation primary response gene (MYD88)
aggatgctggggaactcttt

SEQ ID NO: 13 - DNA, 20 bp, Artificial Sequence
  TCDD-inducible poly(ADP-ribose) polymerase (TIRAP)
tagtgcagcctccttctcct

SEQ ID NO: 14 - DNA, 20 bp, Artificial Sequence
  TCDD-inducible poly(ADP-ribose) polymerase (TIRAP)
aaccccatcaagtgagccag

SEQ ID NO: 15 - DNA, 20 bp, Artificial Sequence
  TNF receptor-associated factor 6, E3 ubiquitin protein ligase (TRAF6)
ggactgcagcaaaagacgac

SEQ ID NO: 16 - DNA, 20 bp, Artificial Sequence
  TNF receptor-associated factor 6, E3 ubiquitin protein ligase (TRAF6)
cttcccgcaaagccatcaag

SEQ ID NO: 17 - DNA, 20 bp, Artificial Sequence
  Interleukin-1 receptor-associated kinase 4 (IRAK4)
acagcatcaacatacgtgcg

SEQ ID NO: 18 - DNA, 20 bp, Artificial Sequence
  Interleukin-1 receptor-associated kinase 4 (IRAK4)
ggtgccccagtcaaacagta

SEQ ID NO: 19 - DNA, 20 bp, Artificial Sequence
  Interleukin-1 receptor-associated kinase 1 (IRAK1)
gccgcccagatctacaagaa

SEQ ID NO: 20 - DNA, 20 bp, Artificial Sequence
  Interleukin-1 receptor-associated kinase 1 (IRAK1)
taggagttctcttgcgggga

SEQ ID NO: 21 - DNA, 20 bp, Artificial Sequence
  V-rel reticuloendotheliosis viral oncogene homolog A (avian) (NF-kappa-B p65 subunit) (RELA)
acagctttcagaacctgggg

SEQ ID NO: 22 - DNA, 19 bp, Artificial Sequence
  V-rel reticuloendotheliosis viral oncogene homolog A (avian) (NF-kappa-B p65 subunit) (RELA)
gacggcattcaggtcgtag

SEQ ID NO: 23 - DNA, 21 bp, Artificial Sequence
  Complement component 3 (C3)
aagttcatcacccacatcaag

SEQ ID NO: 24 - DNA, 20 bp, Artificial Sequence
  Complement component 3 (C3)
cactgtttctggttctcctc

SEQ ID NO: 25 - DNA, 20 bp, Artificial Sequence
  Chemokine (C-C motif) ligand 2 (CCL2)
tctcgctgcaacatgaaggt

SEQ ID NO: 26 - DNA, 20 bp, Artificial Sequence
  Chemokine (C-C motif) ligand 2 (CCL2)
tatagcagcaggcgacttgg

SEQ ID NO: 27 - DNA, 20 bp, Artificial Sequence
  Chemokine (C-C motif) ligand 5 (CCL5)
tccatggcagcagttgtctt

SEQ ID NO: 28 - DNA, 20 bp, Artificial Sequence
  Chemokine (C-C motif) ligand 5 (CCL5)
ttcaggttcaaggcgtcctc

SEQ ID NO: 29 - DNA, 19 bp, Artificial Sequence
  Chemokine (C-C motif) ligand 20 (CCL20)
cttgtgggcttcacacagc

SEQ ID NO: 30 - DNA, 22 bp, Artificial Sequence
  Chemokine (C-C motif) ligand 20 (CCL20)
gtttcacccacttcttctttgg

SEQ ID NO: 31 - DNA, 20 bp, Artificial Sequence
  Chemokine (C-x-C motif) ligand 5 (CXCL5)
ttgtgagagagctgcgttgt

SEQ ID NO: 32 - DNA, 20 bp, Artificial Sequence
  Chemokine (C-x-C motif) ligand 5 (CXCL5)
ccagacagacttcccttcca

SEQ ID NO: 33 - DNA, 25 bp, Artificial Sequence
  Interleukin 8 (CXCL8)
aagaatgagtacagaacttcgatgc

SEQ ID NO: 34 - DNA, 21 bp, Artificial Sequence
  Interleukin 8 (CXCL8)
gtttaggcagacctcgtttcc

SEQ ID NO: 35 - DNA, 20 bp, Artificial Sequence
  Chemokine (C-x-C motif) ligand 3 (CXCL3)
tcaaccctgaagctcccatg

SEQ ID NO: 36 - DNA, 21 bp, Artificial Sequence
  Chemokine (C-x-C motif) ligand 3 (CXCL3)
agtccagcacatcaagtcctt

SEQ ID NO: 37 - DNA, 20 bp, Artificial Sequence
  Chemokine (C-C motif) receptor 7 (CCR7)
atcattgctgtggtcgtggt

SEQ ID NO: 38 - DNA, 20 bp, Artificial Sequence
  Chemokine (C-C motif) receptor 7 (CCR7)
gaaagggttgacacagcagc

SEQ ID NO: 39 - DNA, 20 bp, Artificial Sequence
  Interleukin 13 receptor, alpha 1 (IL13RA)
caggttgaggctggaagaca

SEQ ID NO: 40 - DNA, 20 bp, Artificial Sequence
  Interleukin 13 receptor, alpha 1 (IL13RA)
cccaccactgccatctaagt

SEQ ID NO: 41 - DNA, 20 bp, Artificial Sequence
  Interleukin 1, alpha (IL1-A)
agaatgtggtgatggtggca

SEQ ID NO: 42 - DNA, 20 bp, Artificial Sequence
  Interleukin 1, alpha (IL1-A)
actttgattgagggcgtcgt

SEQ ID NO: 43 - DNA, 20 bp, Artificial Sequence
  Interleukin 1, beta (IL1-B)
gaagaaaggcccgtcttcct

SEQ ID NO: 44 - DNA, 20 bp, Artificial Sequence
  Interleukin 1, beta (IL1-B)
acagtgaagttcaggctgca

SEQ ID NO: 45 - DNA, 21 bp, Artificial Sequence
  Interleukin 6 (IL6)
tggtgatgacttctgctttcc

SEQ ID NO: 46 - DNA, 20 bp, Artificial Sequence
  Interleukin 6 (IL6)
agagcttcggttttctctgg

SEQ ID NO: 47 - DNA, 21 bp, Artificial Sequence
  Interleukin 10 (IL10)
agctgtatccacttgccaacc

SEQ ID NO: 48 - DNA, 21 bp, Artificial Sequence
  Interleukin 10 (IL10)
tgggtcaacagtaagctgtgc

SEQ ID NO: 49 - DNA, 19 bp, Artificial Sequence
  Tumor necrosis factor alpha  (TNFalpha)
ccacgttgtagccgacatc

SEQ ID NO: 50 - DNA, 20 bp, Artificial Sequence
  Tumor necrosis factor alpha  (TNFalpha)
accaccagctggttgtcttc

SEQ ID NO: 51 - DNA, 20 bp, Artificial Sequence
  Transforming growth factor, beta 1 (TGFbeta1)
cctggacaccaactactgct

SEQ ID NO: 52 - DNA, 20 bp, Artificial Sequence
  Transforming growth factor, beta 1 (TGFbeta1)
ccaggaccttgctgtactgt

SEQ ID NO: 53 - DNA, 21 bp, Artificial Sequence
  Serum amyloid A3 (SAA3)
cacgggcatcattttctgctt

SEQ ID NO: 54 - DNA, 20 bp, Artificial Sequence
  Serum amyloid A3 (SAA3)
gggcagcgtcatagtttcca

SEQ ID NO: 55 - DNA, 20 bp, Artificial Sequence
  Haptoglobin (HP)
aatgaacgatggctcctcac

SEQ ID NO: 56 - DNA, 21 bp, Artificial Sequence
  Haptoglobin (HP)
ttgatgagcccaatgtctacc

SEQ ID NO: 57 - DNA, 19 bp, Artificial Sequence
  S100 calcium binding protein A9 (S100A9)
ctggtgcaaaaagagctgc

SEQ ID NO: 58 - DNA, 22 bp, Artificial Sequence
  S100 calcium binding protein A9 (S100A9)
agcataatgaactcctcgaagc

SEQ ID NO: 59 - DNA, 21 bp, Artificial Sequence
  S100 calcium binding protein A12 (S100A12)
tggggaggcgctgctctagac

SEQ ID NO: 60 - DNA, 21 bp, Artificial Sequence
  S100 calcium binding protein A12 (S100A12)
tcgaaatgccccacccgaacg

SEQ ID NO: 61 - DNA, 21 bp, Artificial Sequence
  Lactoferrin (LF)
cgaagtgtggatggcaaggaa

SEQ ID NO: 62 - DNA, 21 bp, Artificial Sequence
  Lactoferrin (LF)
ttcaaggtggtcaagtagcgg

SEQ ID NO: 63 - DNA, 21 bp, Artificial Sequence
  Lysozyme 1 K (LYZ1)
aagaaacttggattggatggc

SEQ ID NO: 64 - DNA, 19 bp, Artificial Sequence
  Lysozyme 1 K (LYZ1)
actgcttttggggttttgc

SEQ ID NO: 65 - DNA, 19 bp, Artificial Sequence
  Lactoperoxidase (LPO)
tggctgtcaaccaagaagc

SEQ ID NO: 66 - DNA, 19 bp, Artificial Sequence
  Lactoperoxidase (LPO)
tgaggctcgaaaatctccc

SEQ ID NO: 67 - DNA, 27 bp, Artificial Sequence
  Tracheal antimicrobial peptide (TAP)
aggagtaggaaatcctgtaagctgtgt

SEQ ID NO: 68 - DNA, 21 bp, Artificial Sequence
  Tracheal antimicrobial peptide (TAP)
agcattttactgcccgcccga

SEQ ID NO: 69 - DNA, 20 bp, Artificial Sequence
  Lingual antimicrobial peptide (LAP)
agaaattctcaaagctgccg

SEQ ID NO: 70 - DNA, 20 bp, Artificial Sequence
  Lingual antimicrobial peptide (LAP)
cagcattttacttgggctcc

SEQ ID NO: 71 - DNA, 20 bp, Artificial Sequence
  Fas cell surface death receptor (FAS)
cgggatctgggttcacttgt

SEQ ID NO: 72 - DNA, 20 bp, Artificial Sequence
  Fas cell surface death receptor (FAS)
ggaggacaaggctgacaaca

SEQ ID NO: 73 - DNA, 20 bp, Artificial Sequence
  Tumor necrosis factor receptor superfamily, member 1A (TNFRSF1A)
cgcctctgtcgtcttagcat

SEQ ID NO: 74 - DNA, 20 bp, Artificial Sequence
  Tumor necrosis factor receptor superfamily, member 1A (TNFRSF1A)
gactggaacttggggtggag

SEQ ID NO: 75 - DNA, 18 bp, Artificial Sequence
  Tumor necrosis factor receptor 2 (TNFR2)
ccagcagcacggacaaga

SEQ ID NO: 76 - DNA, 20 bp, Artificial Sequence
  Tumor necrosis factor receptor 2 (TNFR2)
caatgcaggtgacgttgacc

SEQ ID NO: 77 - DNA, 20 bp, Artificial Sequence
  Caspase 8 (CASP8)
tagcatagcacggaagcagg

SEQ ID NO: 78 - DNA, 21 bp, Artificial Sequence
  Caspase 8 (CASP8)
gccagtgaagtaagaggtcag

SEQ ID NO: 79 - DNA, 20 bp, Artificial Sequence
  Caspase 3 (CASP3)
tcagtcagtcagttgggcac

SEQ ID NO: 80 - DNA, 20 bp, Artificial Sequence
  Caspase 3 (CASP3)
gggagcatcttccacacaca

SEQ ID NO: 81 - DNA, 22 bp, Artificial Sequence
  Caspase 1 (CASP1)
acgtcttgcccttattatctgc

SEQ ID NO: 82 - DNA, 21 bp, Artificial Sequence
  Caspase 1 (CASP1)
gtactgtcagaggtccgatgc

SEQ ID NO: 83 - DNA, 20 bp, Artificial Sequence
  BCL2-associated X protein (BAX)
agaggatgatcgcagctgtg

SEQ ID NO: 84 - DNA, 20 bp, Artificial Sequence
  BCL2-associated X protein (BAX)
gaagtccaatgtccagccca

SEQ ID NO: 85 - DNA, 18 bp, Artificial Sequence
  Anti-apoptotic regulator Bcl-xL (Bcl-xL)
ggcattcagcgacctgac

SEQ ID NO: 86 - DNA, 18 bp, Artificial Sequence
  Anti-apoptotic regulator Bcl-xL (Bcl-xL)
ccatccaagttgcgatcc

SEQ ID NO: 87 - DNA, 20 bp, Artificial Sequence
  B-cell CLL/lymphoma 2 (BCL-2)
atgtgtgtggagagcgtcaa

SEQ ID NO: 88 - DNA, 20 bp, Artificial Sequence
  B-cell CLL/lymphoma 2 (BCL-2)
gagcagtgccttcagagaca

SEQ ID NO: 89 - DNA, 20 bp, Artificial Sequence
  IgG Fc receptor (FcRN)
gagctggctccttggatctc

SEQ ID NO: 90 - DNA, 20 bp, Artificial Sequence
  IgG Fc receptor (FcRN)
ataccaggattcccggaggt

SEQ ID NO: 91 - DNA, 20 bp, Artificial Sequence
  Polymeric immunoglobulin receptor (PIGR)
gacaccgtggagagcaaaga

SEQ ID NO: 92 - DNA, 20 bp, Artificial Sequence
  Polymeric immunoglobulin receptor (PIGR)
gtgattcggagcgtgattgc

SEQ ID NO: 93 - DNA, 19 bp, Artificial Sequence
  CD68 molecule (CD68)
ggctccaaggaggcaatag

SEQ ID NO: 94 - DNA, 20 bp, Artificial Sequence
  CD68 molecule (CD68)
gaatgagaggagcaagtggg

SEQ ID NO: 95 - DNA, 20 bp, Artificial Sequence
  CD40 molecule (CD40)
tcgaaggccaacactgtacc

SEQ ID NO: 96 - DNA, 20 bp, Artificial Sequence
  CD40 molecule (CD40)
gccttttctctcgcagcttg

SEQ ID NO: 97 - DNA, 20 bp, Artificial Sequence
  Signal transducer and activator of transcription 2 (STAT2)
tcctgctgcgctttagtgaa

SEQ ID NO: 98 - DNA, 20 bp, Artificial Sequence
  Signal transducer and activator of transcription 2 (STAT2)
ggattcgcgggtagaggaag

SEQ ID NO: 99 - DNA, 20 bp, Artificial Sequence
  Cytochrome P450, family 1, subfamily B, polypeptide 1 (CYP1B1)
ggactttgacccaacccgat

SEQ ID NO: 100 - DNA, 20 bp, Artificial Sequence
  Cytochrome P450, family 1, subfamily B, polypeptide 1 (CYP1B1)
cactggtgagcaaggatgga

SEQ ID NO: 101 - DNA, 21 bp, Artificial Sequence
  Cytochrome P450, family 1, subfamily A, polypeptide 1 (CYP1A1)
ggagcctaaaacccacagaca

SEQ ID NO: 102 - DNA, 20 bp, Artificial Sequence
  Cytochrome P450, family 1, subfamily A, polypeptide 1 (CYP1A1)
cagcacaactttggaagggc

SEQ ID NO: 103 - DNA, 20 bp, Artificial Sequence
  Nitric oxide synthase 2, inducible (NOS2)
cattcgatgtcagcggcaag

SEQ ID NO: 104 - DNA, 20 bp, Artificial Sequence
  Nitric oxide synthase 2, inducible (NOS2)
gctgcgatttgagcctcatg

SEQ ID NO: 105 - DNA, 20 bp, Artificial Sequence
  FBJ murine osteosarcoma viral oncogene homolog (FOS)
actgctcgcgatcatgatgt

SEQ ID NO: 106 - DNA, 20 bp, Artificial Sequence
  FBJ murine osteosarcoma viral oncogene homolog (FOS)
ccagatcggtgcagtagtcc

SEQ ID NO: 107 - DNA, 20 bp, Artificial Sequence
  Mitogen-activated protein kinase 8 (MAPK8)
tggaggggtaaagggcattg

SEQ ID NO: 108 - DNA, 20 bp, Artificial Sequence
  Mitogen-activated protein kinase 8 (MAPK8)
agaaacggccaggaagtgtt

SEQ ID NO: 109 - DNA, 20 bp, Artificial Sequence
  Matrix metallopeptidase 1 (MMP1)
tctggagcaatgtcacaccc

SEQ ID NO: 110 - DNA, 20 bp, Artificial Sequence
  Matrix metallopeptidase 1 (MMP1)
cctgcacctggttgaaaagc

SEQ ID NO: 111 - DNA, 20 bp, Artificial Sequence
  Interferon regulatory factor 3 (IRF3)
gctcaactgacgggaagtgg

SEQ ID NO: 112 - DNA, 20 bp, Artificial Sequence
  Interferon regulatory factor 3 (IRF3)
tttgggttcccatggtctgg

SEQ ID NO: 113 - DNA, 18 bp, Artificial Sequence
  Myxovirus (influenza virus) resistance 1, interferon-inducible protein p78 (mouse) (MX1)
aaggccactatcccctgc

SEQ ID NO: 114 - DNA, 23 bp, Artificial Sequence
  Myxovirus (influenza virus) resistance 1, interferon-inducible protein p78 (mouse) (MX1)
ctcgtactttggtaaacagtcgg

SEQ ID NO: 115 - DNA, 20 bp, Artificial Sequence
  Myxovirus (influenza virus) resistance 2 (mouse) (MX2)
cttcagagacgcctcagtcg

SEQ ID NO: 116 - DNA, 20 bp, Artificial Sequence
  Myxovirus (influenza virus) resistance 2 (mouse) (MX2)
tgaagcagccaggaatagtg

SEQ ID NO: 117 - DNA, 20 bp, Artificial Sequence
  Nucleotide-binding oligomerization domain containing 2 (NOD2)
ctggctccgaggaaacactt

SEQ ID NO: 118 - DNA, 20 bp, Artificial Sequence
  Nucleotide-binding oligomerization domain containing 2 (NOD2)
gtgctcagatgtcgtcccat

SEQ ID NO: 119 - DNA, 20 bp, Artificial Sequence
  V-akt murine thymoma viral oncogene homolog 1 (AKT1)
gatcaccgacttcggactgt

SEQ ID NO: 120 - DNA, 20 bp, Artificial Sequence
  V-akt murine thymoma viral oncogene homolog 1 (AKT1)
cttctcgtggtcctggttgt

SEQ ID NO: 121 - DNA, 20 bp, Artificial Sequence
  Wingless-type MMTV integration site family member 4 (WNT4)
cggccttcacagtgactctt

SEQ ID NO: 122 - DNA, 20 bp, Artificial Sequence
  Wingless-type MMTV integration site family member 4 (WNT4)
ggcctaggacagtgtttgct

SEQ ID NO: 123 - DNA, 19 bp, Artificial Sequence
  18S ribosomal RNA gene (18SrRNA)
cggggaggtagtgacgaaa

SEQ ID NO: 124 - DNA, 20 bp, Artificial Sequence
  18S ribosomal RNA gene (18SrRNA)
ccgctcccaagatccaacta

SEQ ID NO: 125 - DNA, 20 bp, Artificial Sequence
  H3 histone, family 3A  (H3F3A)
acttgctacaaaagccgctc

SEQ ID NO: 126 - DNA, 20 bp, Artificial Sequence
  H3 histone, family 3A  (H3F3A)
acttgcctcctgcaaagcac

SEQ ID NO: 127 - DNA, 21 bp, Artificial Sequence
  Actin, gamma 1 (ACTG1)
aactccatcatgaagtgtgac

SEQ ID NO: 128 - DNA, 20 bp, Artificial Sequence
  Actin, gamma 1 (ACTG1)
gatccacatctgctggaagg

SEQ ID NO: 129 - DNA, 22 bp, Artificial Sequence
  Glyceraldehyd-3-phosphate dehydrogenase (GAPDH)
gtcttcactaccatggagaagg

SEQ ID NO: 130 - DNA, 20 bp, Artificial Sequence
  Glyceraldehyd-3-phosphate dehydrogenase (GAPDH)
tcatggatgaccttggccag

SEQ ID NO: 131 - DNA, 20 bp, Artificial Sequence
  Tyrosine 3-monoxygenase/tryptophan 5-monoxygenase activation protein, zeta polypeptide (YWHAZ)
caggctgagcgatatgatga

SEQ ID NO: 132 - DNA, 20 bp, Artificial Sequence
  Tyrosine 3-monoxygenase/tryptophan 5-monoxygenase activation protein, zeta polypeptide (YWHAZ)
gaccctccaagatgacctac

SEQ ID NO: 133 - DNA, 20 bp, Artificial Sequence
  Cytokeratin 8 (KRT8)
tggtggaggacttcaagacc

SEQ ID NO: 134 - DNA, 22 bp, Artificial Sequence
  Cytokeratin 8 (KRT8)
cgtgtcagaaatctgagactgc

SEQ ID NO: 135 - DNA, 22 bp, Artificial Sequence
  Ubiquitine B (UBB)
agatccaggataaggaaggcat

SEQ ID NO: 136 - DNA, 19 bp, Artificial Sequence
  Ubiquitine B (UBB)
gctccacctccagggtgat